(19) United States
(12) Patent Application Publication
(10) Pub. No.: US 2005/0187191 A1
(43) Pub. Date: Aug. 25, 2005

(54) Methods and compositions for the treatment of respiratory syncytial virus

(76) Inventors: Louis S. Kucera, Pfafftown, NC (US); Susan L. Morris-Natschke, Apex, NC (US); Khalid S. Ishaq, Chapel Hill, NC (US); Ronald A. Fleming, Cary, NC (US); Jan V. Hess, Hurdle Mills, NC (US); Yunsheng Huang, Apex, NC (US); Russ H. Read, Rural Hall, NC (US); Phillip A. Furman, Durham, NC (US)

Correspondence Address: MORGAN LEWIS & BOCKIUS LLP, 1111 PENNSYLVANIA AVENUE NW, WASHINGTON, DC, 20004, US

(21) Appl. No.: 10/781,894

(22) Filed: Feb. 20, 2004

(51) Int. Cl. A61K 31/685

(52) U.S. Cl. 514/78; 514/114

(57) ABSTRACT

The invention includes compounds useful for inhibiting RSV replication and treating a host infected with RSV. The invention also includes methods of treating a host infected with RSV by administering to the host an anti-RSV effective amount of a compound of the invention.

### 1. FIELD OF THE INVENTION

This invention relates generally to methods and compositions for the treatment of respiratory syncytial viral infections.

### 2. BACKGROUND OF THE INVENTION

Respiratory syncytial virus (RSV) is a negative-strand RNA virus belonging to the family, Paramyxoviridae and to the genus, pneumovirus. The structure and composition of RSV have been described in detail. (Domawchowske et al., Clinical Microbiology Review 12:298-309 (1999)). Two major subgroups of RSV, type A and B, have been identified, as well as antigenic variants within each subgroup (Anderson;, L. J. et al., J Inf Dis 151:626-633 (1985); Mufson, M. A. et al., J Gen Virol 66:2111-2124 (1985)).

RSV is a major human pathogen, responsible for respiratory infection in patients of all ages. Typically, RSV infections remain localized to the upper respiratory tract, causing profuse rhinorrhea, nasal congestion, pharyngitis, cough and fever. In some patients, however, infection spreads to the lower respiratory tract. Severe lower respiratory tract disease (e.g., pneumonia, bronchiolitis) results, and typically requires hospitalization and breathing support. In some circumstances, RSV may be fatal.

Infants and young children, as well as immunocompromised patients and the elderly, are particularly at risk for serious respiratory illness related to RSV infection. Premature infants, as well as young children with chronic heart and lung disease, are at greatest risk for serious complications from RSV infection. Yet, 75% of the hospitalizations for RSV infection occur in infants and children that were previously healthy and without risk factors other than age. Approximately 100,000 children are hospitalized annually in the U.S. with severe cases of pneumonia and bronchiolitis resulting from an RSV infection (Hall, C. B. et al., N Engl J Med 344:1917-1928 (2001)). Moreover, RSV is responsible for hundreds of deaths in infants and young children each year.

RSV is highly contagious. Spread person to person through infected nasal and oral secretions, RSV can also live on surfaces for hours. Typically, RSV occurs in epidemics that last up to 4 months, from late fall through early spring. Children in day-care centers and preschools are at significant risk, and the elderly in hospitals and nursing homes are particularly vulnerable. School-aged children are commonly implicated in the spread of the disease, both to their younger siblings and their parents.

It has been recently reported that RSV accounts for 13.5-21.6% of the $2.25 billion in costs associated with hospitalization of infants with lower respiratory infections. The total costs are even greater, as these include treatment for other populations at high risk for RSV, including the elderly and the immunocompromised. RSV also affects healthy adults, and even in a milder form is associated with significant work absences. Re-infection with RSV is very common, forcing additional costs on the healthcare system. The health-related effects of initial infection in some populations, moreover, may be long term. Recent data indicates that RSV-induced lower respiratory tract infections in infants may be linked to the development of asthma or reactive airway disease in later childhood (Sigurs, N. et al., Am J Resp Crit Care Med 161:1501-1507 (2000)).

Despite the prevalence of RSV infection, and significant advances in scientific understanding, there are few approved treatments and even more controversy surrounding the proper management of high-risk populations. In general, most treatments for RSV are symptomatic. Supportive therapy may include monitoring, removal of secretions, intravenous hydration, administration of oxygen. Bronchodilators (e.g., metaproterenol or albuterol) and corticosteroids may be used, but most studies have concluded that there is insufficient evidence of therapeutic benefit except, perhaps, in certain subgroups (i.e., infants). Studies on vitamin A and interferon have also yielded disappointing results. Exogenous surfactant administration may be helpful in improving oxygenation in infants with severe RSV respiratory infection (Greenough, Acta Paediatr. Suppl. 2001, 436: 11-14).

Anti-viral therapy for RSV is limited to a single FDA-approved agent, Ribavirin (Virazole®). Ribavirin (1-beta-D-ribafuranosyl-1,2,4-traizole-3-carboxamide) is a synthetic nucleoside analog with broad spectrum antiviral activity against RSV, influenza, parainfluenza, adenovirus, measles, Lassa fever, and Hantaan viruses. Ribavirin is FDA-approved for use in very ill children hospitalized with severe RSV-related pneumonia. Yet, the use of Ribavirin even for this limited purpose is controversial in view of its mixed clinical history, cost and difficulty of administration and potential safety issues for secondary exposure. Extensive clinical trials on Ribavirin have generally failed to show that the drug reduces hospitalization time, length or severity of RSV bronchiolitis, or need for supportive therapies or mortality. As a result, routine use of Ribavirin, even in high-risk children with RSV infections, is no longer warranted.

Unlike treatment, prevention of RSV disease has been largely successful (Kimpen et al., Respiratory Research Vol. 3, Suppl 1, pp. S40-45 (2002)). Prevention is addressed primarily through passive immunization strategies that provide temporary immunity. The use of hyperimmune globulins such as RSV-IG (Respigam®; Medlmmune, Inc.) for the prevention of RSV in high-risk infants has been approved by the FDA. Drawbacks associated with the use of Respigam® include the long duration of intravenous administration (e.g., 6 hours), the considerable volume required (15 ml/kg), possible interference with normal vaccinations and high cost. The humanized murine monoclonal antibody palivizumab (Synagis®; Medlmmune, Inc.) has also been approved for the prevention of RSV lower respiratory tract disease. It is considerably easier to administer than RSV-IG (i.e., by monthly intramuscular injection) and doesn't interfere with normal vaccination, but it is also very costly.

While passive immunization has proven effective as a preventive for high-risk patients, most patients that develop RSV disease were previously healthy and did not exhibit risk factors other than age. There is currently no known vaccine for RSV. Challenges to development of a vaccine include, among others: (1) the possibility that vaccination will potentiate naturally occurring RSV disease; (2) the inability of many high-risk patients (e.g., newborns and very young children) to mount a protective immune response; and (3) the need to provide protection against multiple antigenic strains of RSV. Strategies for developing an RSV vaccine have included inactivated virus (e.g., formalin-inactivated vaccine), live-attenuated viruses (e.g., cold-adapted and/or temperature sensitive vaccine) and subunit vaccines (e.g., RSV F subunit vaccine).

U.S. Pat. No. 5,962,437 to Kucera et al. discloses methods of treating viral infections, in particular, HIV-1, hepatitis V virus, and herpes viruses. The compounds disclosed in U.S. Pat. No. 5,962,437 are phospholipids or phospholipid derivatives substituted at the 1-position with a C₆-C₁₈ alkyl group and at the 2-position with a C₆-C₁₄ alkyl group. The patent suggests that the compounds disclosed therein could also be used to treat RSV. However, given the complexities of the mechanism of replication of RSV and the pathology of RSV, compared with the other viruses listed in the patent, until the present invention, the successful treatment of RSV with the disclosed compounds could not have been predicted.

In light of the fact that RSV remains a serious public health threat, there remains a strong need to provide new effective pharmaceutical agents to treat humans infected with the virus. The ideal new pharmaceutical agent would be potent, long-acting and easy to administer. It is therefore an object of the present invention to provide new methods for the treatment of human patients and other hosts infected with RSV.

### 3. SUMMARY OF THE INVENTION

The invention is directed to compounds of Formula I: 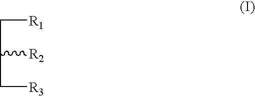
and pharmaceutically acceptable salts or prodrugs thereof,
wherein:
R₁ is —NHC(O)Y, where Y is C₁-C₂₂ alkyl, C₂-C₂₂ alkenyl, or C₂-C₂₂ alkynyl;
R₂ is —OX, where X is C₁-C₂₂ alkyl, C₂-C₂₂ alkenyl, C₂-C₂₂ alkynyl; and
R₃ is phosphocholine.

In one embodiment of the compound of formula I, Y and X are independently C₁-C₁₄ alkyl, C₂-C₁₄ alkenyl, or C₂-C₁₄ alkynyl.

In one embodiment of the compound of formula I, Y is —C₁₀H₂₁; and X is —CH₂CH₃, —CH₂CH₂CH₃, —CH₂CH₂CH₂CH₃, or —C₁₀H₂₁.

In one embodiment of the compound of formula I, Y is —C₁₁H₂₃ and X is C₁-C₅ alkyl

In one embodiment of the compound of formula I, Y is —C₉H₁₉ and X is C₉-C₁₁ alkyl.

In one embodiment the compound of formula I is 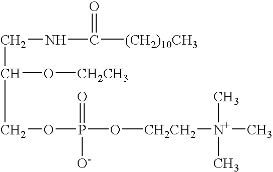
3-dodecanamido-2-ethoxypropyl-1-phosphocholine, 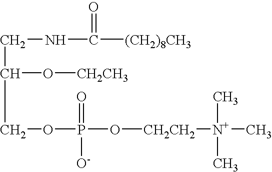
3-decanamido-2-ethoxypropyl-1-phosphocholine, 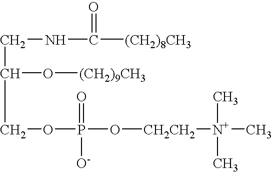
3-decanamido-2-decyloxypropyl-1-phosphocholine, 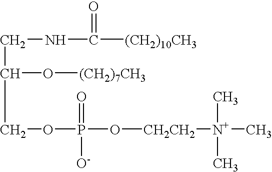
3-dodecanamido-2-octyloxypropyl-1-phosphocholine, 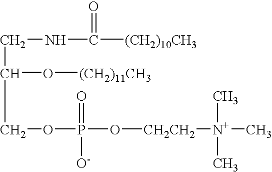
3-dodecanamido-2-dodecyloxypropyl-1-phosphocholine, or 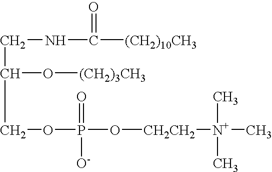
3-dodecanamido-2-butyloxy-1-phosphocholine.

The invention is also directed a compound of Formula II: 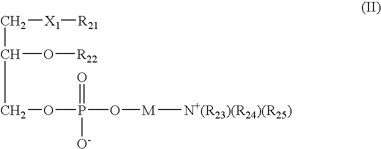
and pharmaceutically acceptable salts or prodrugs thereof,
wherein:
M is C₂-C₄ alkyl;
X₁ is —S—, —O—, —NH—, or —NHC(O)—;
R₂₁ is C₁-C₂₀ straight chain alkyl, C₂-C₂₀ straight chain alkylene containing not more than four double bonds, or aryl;
R₂₂ is C₁-C₂₀ straight chain alkyl, C₂-C₂₀ straight chain alkylene containing not more than four double bonds, or aryl; and
R₂₃, R₂₄, and R₂₅ are each independently hydrogen, methyl, ethyl, propyl, or isopropyl.

In one embodiment, of the compound of Formula II,
M is —CH₂CH₂—; X₁ is —NHC(O)—;
R₂₁ is a C₁-C₁₆ straight chain alkyl or C₂-C₁₆ straight chain alkylene containing not more than one double bond;
R₂₂ is a C₁-C₁₆ straight chain alkyl or C₂-C₁₆ straight chain alkylene containing not more than one double bond; and
R₂₃, R₂₄, and R₂₅ are each independently hydrogen or methyl.

In one embodiment, of the compound of Formula II,
R₂₁ is a C₁-C₁₆ straight chain alkyl or C₂-C₁₆ straight chain alkylene containing not more than one double bond; and
R₂₂ is a C₁-C₅ straight chain alkyl or C₂-C₅ straight chain alkylene containing not more than one double bond.

In one embodiment, of the compound of Formula II, R₂₁ is C₉-C₁₂ alkyl and R₂₂ is C₁-C₁₂ alkyl.

In one embodiment, of the compound of Formula II, R₂₁ is C₉-C₁₂ alkyl and R₂₂ is C₁-C₅ alkyl.

In one embodiment, of the compound of Formula II, R₂₁ is C₉-C₁₂ alkyl and R₂₂ is C₈-C₁₂ alkyl.

The invention is also directed to a compound of Formula III: 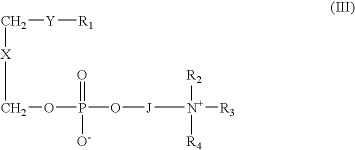
and pharmaceutically acceptable salts or prodrugs thereof,
wherein:
Y is —S—, —O—, —NH—, —N(CH₃)—, —NHC(O)—, or —N(CH₃)C(O)—;
R₁ is C₁-C₁₈ alkyl, C₂-C₁₈ alkenyl, C₂-C₁₈ alkynyl, or aryl;
X is a covalent bond or methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl), or —C(O)N(C₂-C₂₀ alkynyl);
J is a C₁-C₄ alkyl optionally substituted from one to three times with methyl or ethyl; and
R₂, R₃, and R₄ are independently hydrogen or C₁-C₃ alkyl.

In one embodiment, of the compound of Formula III,
Y is —NHC(O)—;
R₁ is C₆-C₁₈ alkyl;
X is —C(H)(O—C₁-C₁₈ alkyl)- or —C(H)(O—C₁-C₁₈ alkenyl)-;
J is —CH₂CH₂—; and
R₂, R₃, and R₄ are each methyl.

In one embodiment, of the compound of Formula III, R₁ is —C₁₁H₂₃ and X is —C(H)(O—C₁-C₅ alkyl)- or —C(H)(O—C₁-C₅ alkenyl)-

In one embodiment, of the compound of Formula III, R₁ is —C₉H₁₉ and X is —C(H)(OC₂H₅)—

In one embodiment, of the compound of Formula III, R₁ is —C₉H₁₉ and X is —C(H)(OC₁₀H₂₁)—.

The invention is also directed to a compound of Formula IV: 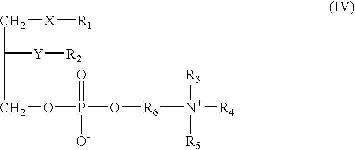
and pharmaceutically acceptable salts or prodrugs thereof,
wherein:
R₁ is a C₆-C₁₈ alkyl, C₆-C₁₈ alkenyl, or C₆-C₁₈ alkynyl that is optionally substituted from 1 to 5 times with —OH, —COOH, oxo, amino, or aryl;
X is —NHC(O)—, —N(CH₃)C(O)—, —C(O)NH—, —C(O)N(CH₃)—, —S—, —S(O)—, —(SO₂)—, —O—, —NH—, and —N(CH₃)—;
R₂ is a C₁-C₁₄ alkyl, C₂-C₁₄ alkenyl, or C₂-C₁₄ alkynyl that is optionally substituted from 1 to 5 times with —OH, —COOH, oxo, amino, or aryl;
Y is —NHC(O)—, —N(CH₃)C(O)—, —C(O)NH—, —C(O)N(CH₃)—, —S—, —S(O)—, —(SO₂)—, —O—, —NH—, —N(CH₃)—, or —OC(O)—;
R₆ is a C₂-C₆ alkyl; C₂-C₆ alkenyl, or C₂-C₆ alkynyl; and
R₃, R₄, and R₅ are independently methyl or ethyl, or R₃ and R₄ together form an aliphatic or heterocyclic ring having five or six ring atoms and R₅ is methyl or ethyl.

In one embodiment, of the compound of Formula IV,
R₂ is C₁-C₁₄ alkyl, C₂-C₁₄ alkenyl, or C₂-C₁₄ alkynyl,;
R₆ is —CH₂CH₂—; and
R₃, R₄, and R₅ are each independently CH₃.

In one embodiment, of the compound of Formula IV, R₂ is C₁-C₅ alkyl or C₂-C₅ alkenyl.

In one embodiment, of the compound of Formula IV, R₁ is C₈-C₁₂ alkyl and R₂ is C₁-C₁₂ alkyl.

In one embodiment, of the compound of Formula IV, R₁ is C₈-C₁₂ alkyl and R₂ is C₁-C₅ alkyl.

In one embodiment, of the compound of Formula IV, R₁ is C₈-C₁₂ alkyl and R₂ is C₈-C₁₂ alkyl

In one embodiment, of the compound of Formula IV,
X is —NHC(O), —N(CH₃)C(O)—, —C(O)NH—, —C(O)N(CH₃) and
Y is —O—, —NH—, or —N(CH₃)—.

The invention is also directed to a compound of Formula AA-1: 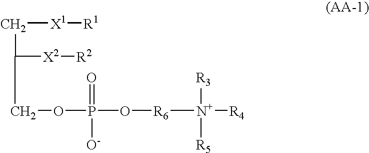
and pharmaceutically acceptable salts or prodrugs thereof,
wherein:
X₁ is —NHC(O)—;
X₂ is —O—;
R₁ is —C₁-C₂₂ alkyl;
R is —C₁-C₂₂ alkyl;
R⁶ is —CH₂CH₂—; and
R³, R⁴, and R⁵ are methyl.

In one embodiment, of the compound of Formula AA-1,
R¹ is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃; and
R² is —CH₃, —CH₂CH₃, —CH₂CH₂CH₃, —CH₂CH₂CH₂CH₃, —CH₂CH₂CH₂CH₂Ch₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃.

In one embodiment, of the compound of Formula AA-1,
R¹ is —(CH₂)₈CH₃, —(CH₂)₉CH₃, —(CH₂)₁₀CH₃, —(CH₂)₁₁CH₃; —(CH₂)₁₂CH₃, or —(CH₂)₁₃CH₃; and
R² is CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, or —(CH₂)₇CH₃.

In one embodiment, of the compound of Formula AA-1,
R¹ is —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, or —(CH₂)₁₂CH₃; and
R² is —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, or —(CH₂)₁₃CH₃.

The invention also provides a method for treating a host infected with RSV. This method comprises administering to a host in need thereof an anti-RSV effective amount of compound of Formula I-IV or AA-1, or a pharmaceutically acceptable salt or prodrug thereof. In one embodiment, the host is a mammal. In one embodiment, the host is a human.

The compound of Formula I-IV or AA-1, or a pharmaceutically acceptable salt or prodrug thereof can be administered orally, intravenously, parentally, intradermally, subcutaneously, topically, or by inhalation.

The invention further provides a method of inhibiting RSV replication in a cell. This method comprises administering to the cell, in an amount effective to inhibit replication of RSV in a cell, a compound of Formula I-IV or AA-1, or a pharmaceutically acceptable salt or prodrug thereof. In one embodiment, the host is a mammal. In one embodiment, the host is a human.

The invention also provides pharmaceutical compositions comprising a compound of Formula I-IV or AA-1, or a pharmaceutically acceptable salt or prodrug thereof.

The invention also provides a kit comprising a compound of Formula I-IV or AA-1, or a pharmaceutically acceptable salt or prodrug thereof.

The invention also provides the use of a compound of Formula I -IV or AA-1, or a pharmaceutically acceptable salt or prodrug thereof, optionally with a pharmaceutical acceptable carrier or diluent, for the manufacture of a medicament for the treatment of a host infected with RSV.

### 4. BRIEF DESCRIPTION OF THE DRAWINGS

FIG. 1 illustrates a process that may be used generally for obtaining a 3-alkylamido-2-alkoxypropylphosphocholine.

### 5. DETAILED DESCRIPTION OF THE INVENTION

Provided are methods and compositions for the treatment of RSV infection in humans and other host animals. The compounds useful for the treatment of RSV infection are alkylamidophosphocholine compounds or analogs thereof and pharmaceutically acceptable salts or prodrugs thereof (hereinafter “compounds of the invention”). The methods involve administering an effective amount of a compound of the invention, optionally in a pharmaceutically acceptable carrier, to a host in need thereof. The compounds of the invention can be used singly or in combination. The methods treat or retard the progression of clinical illness in an individual infected with RSV. The methods of treatment can be used to treat severe RSV lower respiratory tract infections including, but not limited to, the treatment of RSV bronchiolitis and pneumonia.

The invention also includes a method of inhibiting RSV replication in a cell. This method comprises administering to the cell, in an amount effective to inhibit replication of RSV in a cell, a compound of the invention.

Compounds of the invention that can be administered include, but are not limited to, 3-dodecanamido-2-ethoxypropyl-1-phosphocholine, 3-decanamido-2-ethoxypropyl-1-phosphocholine, 3-decanamido-2-decyloxypropyl-1-phosphocholine, 3-dodecanamido-2-octyloxypropyl-1-phosphocholine, 3-dodecanamido-2-dodecyloxypropyl-1-phosphocholine, 3-dodecanamido-2-butyloxy-1-phosphocholine, optionally in a pharmaceutically acceptable carrier. The compounds may possess anti-RSV activity or be metabolized to a compound or compounds that exhibit anti-RSV activity. Without wishing to be bound by theory, it is believed that the effectiveness of the compounds of the invention may be due to the fact that they are phosphocholine (PC) analogs which may provide a surfactant effect to assist in the removal of pulmonary secretions and improve oxygenation that is beneficial against RSV if administered by pulmonary administration.

Previous studies have established that a PC moiety is an essential component for a phospholipid to exhibit optimal antiviral activity (Piantadosi et al., 1991, J. Med. Chem. 34:1408-1414; Krugner-Higby et al., 1995, AIDS Res. & Human Retrovir. 11:705-712). Lipid compounds comprising phosphatidic acid, phosphoethanolamine, phosphoalkylpyridine, alcohol or quarternary amine salt moieties were less active, more toxic, exhibited much lower differential selectivities or some combination of these, relative to the corresponding PC lipids. In the compounds of the invention, a PC moiety is incorporated into the lipid backbone to provide compounds that exhibit optimal antiviral activity.

### 5.1 DEFINITIONS

As used herein, RSV infection and related conditions include, but are not limited to, one or more conditions, such as acute respiratory illness, pneumonia, bronchiolitis (inflammation of the small airways of the lungs), tracheobronchitis (croup), and ostitis media (ear infections).

The term “treatment” as used herein, means an approach for obtaining beneficial or desired results including clinical results, such as, but not limited to, alleviation of symptoms of a disease or condition, diminishment of extent of a disease or condition, stabilization (i.e., not worsening) of a disease or condition, preventing spread of a disease or condition, preventing or reducing occurrence or recurrence of a disease or condition, delaying or slowing of a disease's or condition's progression, and reducing the incidence of a disease or condition or the symptoms of a disease or condition.

The articles “a” and “an” are used herein to refer to one or more than one (i.e., at least one) of the grammatical object of the article. By way of example, “an element” means on element or more than one element.

Compounds of the invention having a chiral center can exist in and be isolated in distinct optically active or racemic forms. The present invention encompasses racemic, optically active, and stereoisomeric forms, and all mixtures of such forms of a compound of the invention. Methods of preparing optically active forms of a compound are well known in the art and include, for example, resolution of a racemic mixture of the compound using recrystallization techniques, synthesis of the compound from optically active starting materials, chiral synthesis of the compound, and chromatographic separation of a racemic micture of the compound using a chiral stationary phase. The present invention also encompasses polymorphic forms and mixtures thereof.

Determining or assessing antiviral activity may be performed using standard tests described herein or other tests know in the art.

As used herein, the term “alkyl,” unless otherwise specified, means a saturated straight chain or branched, acyclic or cyclic, primary, secondary, or tertiary hydrocarbon. Examples of alkyl include, but are not limited to, methyl, ethyl, propyl, isopropyl, butyl, iso-butyl, sec-butyl, pentyl, sec-pentyl, iso-pentyl, hexyl, iso-hexyl, sec-hexyl, heptyl, sec-heptyl, iso-heptyl, octyl, iso-octyl, sec-octyl, nonyl, iso-nonyl, sec-nonyl, undecyl, iso-undecyl, sec-undecyl, dodecyl, iso-dodecyl, sec-dodecyl, tridecyl, iso-tridecyl, sec-tridecyl, tetradecyl, iso-tetradecyl, sec-tetradecyl, pentadecyl, iso-pentadecyl, sec-pentadecyl, and eicosyl moieties.

As used herein, the term “C₁-C₃ alkyl” means a saturated straight chain or branched, acyclic or cyclic, primary, secondary, or tertiary hydrocarbon having from 1 to 3 carbon atoms.

As used herein, the term “C₁-C₄ alkyl” means a saturated straight chain or branched, acyclic or cyclic, primary, secondary, or tertiary hydrocarbon having from 1 to 4 carbon atoms.

As used herein, the term “C₂-C₄ alkyl” means a saturated straight chain or branched, acyclic or cyclic, primary, secondary, or tertiary hydrocarbon having from 2 to 4 carbon atoms.

As used herein, the term “C₂-C₆ alkyl” means a saturated straight chain or branched, acyclic or cyclic, primary, secondary, or tertiary hydrocarbon having from 2 to 6 carbon atoms.

As used herein, the term “C₆-C₁₈ alkyl” means a saturated straight chain or branched, acyclic or cyclic, primary, secondary, or tertiary hydrocarbon having from 6 to 18 carbon atoms.

As used herein, the term “C₂-C₁₄ alkyl” means a saturated straight chain or branched, acyclic or cyclic, primary, secondary, or tertiary hydrocarbon having from 2 to 14 carbon atoms.

As used herein, the term “C₁-C₂₂ alkyl” means a saturated straight chain or branched, acyclic or cyclic, primary, secondary, or tertiary hydrocarbon having from 1 to 22 carbon atoms.

As used herein, the term “C₁-C₂₀ alkyl” means a saturated straight chain or branched, acyclic or cyclic, primary, secondary, or tertiary hydrocarbon having from 1 to 20 carbon atoms.

As used herein, the term “C₁₄-C₁₈ alkyl” means a saturated straight chain or branched, acyclic or cyclic, primary, secondary, or tertiary hydrocarbon having from 14 to 18 carbon atoms.

As used herein, the term “C₉-C₃₀ alkyl” means a saturated straight chain or branched, acyclic or cyclic, primary, secondary, or tertiary hydrocarbon having from 9 to 30 carbon atoms.

As used herein, the term “alkenyl,” unless otherwise specified, means a straight chain or branched, acyclic or cyclic, hydrocarbon having at least 2 carbon atoms and including at least one carbon-carbon double bond. Examples of alkenyl include, but are not limited to, vinyl, allyl, 1-butenyl, 2-butenyl, isobutenyl, 1-pentenyl, 2-pentenyl, 3-methyl-1-butenyl, 2-methyl-1-butenyl, 2,3-dimethyl-2-butenyl, 1-hexenyl, 2-hexenyl, 3-hexenyl, 2-heptenyl, 3-heptenyl, 1-octenyl, 2-octenyl, 3-octenyl, 1-nonenyl, 2-nonenyl, 3-nonenyl, 1-decenyl, 2-decenyl, and 3-decenyl moieties.

As used herein, the term “C₂-C₂₂ alkenyl” means a straight chain or branched, acyclic or cyclic, primary, secondary, or tertiary hydrocarbon having from 2 to 22 carbon atoms and including at least one carbon-carbon double bond.

As used herein, the term “C₂-C₂₀ alkenyl” means a straight chain or branched, acyclic or cyclic, primary, secondary, or tertiary hydrocarbon having from 2 to 20 carbon atoms and including at least one carbon-carbon double bond.

As used herein, the term “C₆-C₁₈ alkenyl” means a straight chain or branched, acyclic or cyclic, primary, secondary, or tertiary hydrocarbon having from 6 to 18 carbon atoms and including at least one carbon-carbon double bond.

As used herein, the term “C₂-C₁₄ alkenyl” means a straight chain or branched, acyclic or cyclic, primary, secondary, or tertiary hydrocarbon having from 2 to 14 carbon atoms and including at least one carbon-carbon double bond.

As used herein, the term “C₉-C₃₀ alkenyl” means a saturated straight chain or branched, acyclic or cyclic, primary, secondary, or tertiary hydrocarbon having from 9 to 30 carbon atoms and including at least one carbon-carbon double bond.

As used herein, the term “alkynyl,” unless otherwise specified, means a straight chain or branched, acyclic hydrocarbon having at least 2 carbon atoms and including at least one carbon-carbon triple bond. Examples of alkynyl include, but are not limited to, acetylenyl, propynyl, 1-butynyl, 2-butynyl, 1-pentynyl, 2-pentynyl, 3-methyl-1-butynyl, 4-pentynyl, 1-hexynyl, 2-hecynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, and 9-decynyl moieties.

As used herein, the term “C₂-C₂₂ alkynyl” means a straight chain or branched, acyclic primary, secondary, or tertiary hydrocarbon having from 2 to 22 carbon atoms and including at least one carbon-carbon triple bond.

As used herein, the term “C₂-C₂₀ alkynyl” means a straight chain or branched, acyclic primary, secondary, or tertiary hydrocarbon having from 2 to 20 carbon atoms and including at least one carbon-carbon triple bond.

As used herein, the term “C₆-C₁₈ alkynyl” means a straight chain or branched, acyclic primary, secondary, or tertiary hydrocarbon having from 6 to 18 carbon atoms and including at least one carbon-carbon triple bond.

As used herein, the term “C₂-C₁₄ alkynyl” means a straight chain or branched, acyclic primary, secondary, or tertiary hydrocarbon having from 2 to 14 carbon atoms and including at least one carbon-carbon triple bond.

As used herein, the term “C₉-C₃₀ alkynyl” means a saturated straight chain or branched, acyclic, primary, secondary, or tertiary hydrocarbon having from 9 to 30 carbon atoms and at least on carbon-carbon triple bond.

As used herein, the term “aryl,” unless otherwise specified, means phenyl, biphenyl, or napthyl, optionally substituted with one or more of halo, alkyl, alkenyl, alkynyl, —OH, —NH₂, —NHR¹, —NR¹R¹, —NH(aryl), —NH(aryl)(aryl), —O-alkyl, —O-alkenyl, —O-alkynyl, —O-aryl, nitro, cyano, —S-alkyl, —S-alkenyl, —S-alkynyl, —S-aryl, —NR¹C(O)R¹, —COOH, —SO₃H, —COOR¹, —OP(O)(OR¹)₂, —OP(O)(R¹)(OR¹), —OP(O)(R¹)₂, either unprotected or protected using a protecting group (as known to those skilled in the art, for example, as taught in Greene et al. Protective Groups in Organic Synthesis. Jogn Wiley and Sons, 2^{nd} edition (1991)), wherein each R¹ is independently hydrogen, alkyl, alkenyl, or alkynyl.

As used herein, the term “halo” or “halogen” as used herein means —Cl, —Br, —I, and —F.

As used herein, the term “amino” means —NH₂.

As used herein, the term “oxo” means a methylene group wherein the two hydrogens of the methylene are replaced with double bond to oxygen.

As used herein, the term “heterocyclic ring,” unless otherwise specified, means a 3 to 10 membered monocyclic or bicyclic ring which is either saturated, unsaturated non-aromatic, or aromatic containing from 1 to 4 heteroatoms independently selected from nitrogen, which can be quaternized; oxygen; and sulfur, including sulfoxide and sulfone. The heterocycle ring can be attached by a nitrogen, sulfur, or carbon atom. Representative heterocycles include, but are not limited to, pyridyl, furyl, thiophenyl, pyrrolyl, oxazolyl, imidazolyl, thiazolyl, thiadiazolyl, isooxazolyl, pyrazolyl, isothiazolyl, pyridazinyl, pyrimidinyl, pyrazinyl, triazinyl, morpholinyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, quinolinyl, isoquinolinyl, chromonyl, coumarinyl, indolyl, indolizinyl, benzo[b[furanyl, benzo[b]thiophenyl, indazolyl, purinyl, 4H-quinolizinyl, isoquinolyl, quinolyl, phthalazinyl, naphthyridinyl, and carbazolyl.

As used herein, the term “heteroaromatic,” unless otherwise specified, means an aromatic heterocycle ring having between 5 and 10 ring atoms, including both monocyclic and bicyclic ring systems, wherein at least one carbon atom of one or both of the rings is replaced with a heteroatom independently selected from nitrogen, oxygen, and sulfur. Representative heteroaromatics include, but are not limited to, pyridyl, furyl, benzofutanyl, thiophenyl, benzothiophenyl, quinolynyl, pyrrolyl, indolyl, oxazolyl, benzooxazolyl, imidazolyl, benzimidazolyl, thiazolyl, benzothiazolyl, isoxazolyl, pyrazolyl, isothiazolyl, pyridazinyl, pyrimidinyl, pyrazinyl, thiadiazolyl, triazinyl, cinnolinyl, phthalazinyl, and quinazolinyl.

As used herein the term “cycloalkane ring” or “cycloalkyl,” unless otherwise specified, means a 3 to 14 membered monocyclic, bicyclic, or tricyclic hydrocarbon ring which is either saturated or unsaturated non-aromatic. Representative cycloalkane rings include, but are not limited to, cyclopropyl, cyclobutyl, cyclopentyl, cyclohexyl, cycloheptyl, cyclooctyl, indanyl, 1,2,3,4-tetrahydronaphthyl, perhydronaphthyl, 1,2,3,4-tetrahydroanthracenyl, cyclopentenyl, cyclopentadienyl, cyclohexenyl, cycloheptenyl, cyclohetadienyl, and cycloheptatrienyl.

As used herein the term “C₃-C₈ cycloalkyl” or “C₃-C₈ cycloalkane ring” means a 3 to 8 membered monocyclic hydrocarbon ring a which is either saturated or unsaturated non-aromatic. Representative C₃-C₈ cycloalkane rings include, but are not limited to, cyclopropyl, cyclobutyl, cyclopentyl, cyclohexyl, cycloheptyl, and cyclooctyl.

As used herein, the term “prodrug” means a compound that, when administered to an animal, is converted under physiological conditions to a compound of the invention.

As used herein, the term “anti RSV effective amount” means and amount effective for treating RSV.

The term “host”, as used herein, refers to a unicellular or multicellular organism in which the virus can replicate, including cell lines and animals, and preferably a human. Alternatively, the host can be carrying a part of the viral genome, whose replication or function can be altered by the compounds of the invention. The term host refers to infected cells, cells transfected with all or part of the RSV genome, and animals, in particular, primates (including chimpanzees) and humans. In most methods of the invention, the host is a human patient. Veterinary applications, in certain indications, however, are clearly encompassed by the present invention, such as in chimpanzees.

The term “pharmaceutical salt” refers to a salt that retains the desired biological activity of the parent compound and preferably does not impart undesired toxicological effects thereto. Examples of salts include, but are not limited to, (a) salts formed with cations such as sodium, potassium, NH₄⁺, magnesium, and calcium polyamines such as spermine and spermidine; (b) acid addition salts formed with inorganic acids including, but not limited to, hydrochloric acid, hydrobromic acid, sulfuric acid, phosphoric acid, and nitric acid; (c) salts formed with organic acids including, but not limited to, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid; and (d) salts formed from elemental anions such as chloride, bromide, and iodide.

### 5.2 COMPOUNDS OF THE INVENTION

A variety of compounds may be used in the methods disclosed herein for the treatment of RSV. These compounds exhibit anti-RSV activity.

### 5.2.1 COMPOUNDS OF FORMULA I

The compounds used in the methods of the invention include compounds of Formula I: 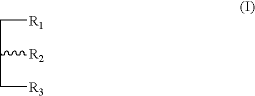
and pharmaceutically acceptable salts or prodrugs thereof
wherein:
R₁ is —NHC(O)Y, where Y is C₁-C₂₂ alkyl, C₂-C₂₂ alkenyl, or C₂-C₂₂ alkynyl;
R₂ is —OX, where X is C₁-C₂₂ alkyl, C₂-C₂₂ alkenyl, or C₂-C₂₂ alkynyl; and
R₃ is phosphocholine (—OPO₃⁻CH₂CH₂N⁺(CH₃)₃).

In one embodiment, Y is C₁-C₂₂ alkyl.

In one embodiment, Y is C₂-C₂₂ alkenyl.

In one embodiment, Y is C₂-C₂₂ alkynyl.

In one embodiment, X is C₁-C₂₂ alkyl.

In one embodiment, X is C₂-C₂₂ alkenyl.

In one embodiment, X is C₂-C₂₂ alkynyl.

In one embodiment, X is a C₁-C₅ alkyl.

In one embodiment, X is C₂-C₅ alkenyl.

In one embodiment, X is C₂-C₅ alkynyl.

In one embodiment, X is —C₂H₅ or —C₁₀H₂₁.

In one embodiment, X is —C₂H₅.

In one embodiment, Y is —C₉H₁₉ or —C₁₁H₂₃.

In one embodiment, Y is —C₁₁H₂₃, X is —C₂H₅, and R₃ is phosphocholine.

In one embodiment, Y is —C₉H₁₉, X is —C₂H₅, and R₃ is phosphocholine.

In one embodiment, Y is —C₉H₁₉, X is —C₁₀H₂₁, and R₃ is phosphocholine.

### 5.2.2 COMPOUNDS OF FORMULA II

The compounds used in the methods of the invention also include compounds of Formula II: 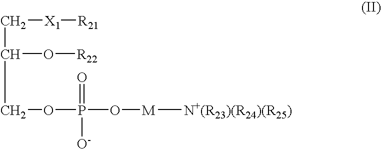
and pharmaceutically acceptable salts or prodrugs thereof,
wherein:
M is a C₂-C₄ alkyl;
X₁ is —S—, —O—, —NH—, or —NHC(O)—;
R₂₁ is a C₁-C₂₀ straight chain alkyl, C₂-C₂₀ straight chain alkylene containing not more than four double bonds, or aryl;
R₂₂ is a hydrogen, C₁-C₂₀ straight chain alkyl, or C₂-C₂₀ straight chain alkylene containing not more than four double bonds; and
R₂₃, R₂₄, and R₂₅ are each independently either hydrogen, methyl, ethyl, propyl, or isopropyl.

In one embodiment, M is —CH₂CH₂—.

In one embodiment, M is —CH₂CH₂CH₂—.

In one embodiment, M is —CH₂CH₂CH₂CH₂—.

In one embodiment, M is —CH₂CH(CH₃)—

In one embodiment, M is —CH(CH₃)CH₂—.

In one embodiment, M is —CH(CH₃)CH₂CH₂—.

In one embodiment, M is —CH₂CH(CH₃)CH₂—.

In one embodiment, M is —CH₂CH₂CH(CH₃)—.

In one embodiment, M is —C(CH₃)₂—.

In one embodiment, M is —CH₂C(CH₃)₂—.

In one embodiment, M is —C(CH₃)₂CH₂—.

In one embodiment, X₁ is —S—.

In one embodiment, X₁ is —O—.

In one embodiment, X₁ is —NH—.

In one embodiment, X₁ is —NHC(O)—.

In one embodiment, R₂₁ is a C₁-C₂₀ straight chain alkyl.

In one embodiment, R₂₁ is a straight chain C₂-C₂₀ alkylene containing not more than four double bonds.

In one embodiment, R₂₁ is aryl.

In one embodiment, R₂₂ is a C₁-C₂₀ straight chain alkyl.

In one embodiment, R₂₂ is a C₂-C₂₀ straight chain alkylene containing not more than four double bonds.

In one embodiment, R₂₂ is a C₁-C₅ straight chain alkylene containing not more than four double bonds.

In one embodiment, R₂₂ is a C₁-C₅ straight chain alkyl.

In one embodiment, R₂₂ is hydrogen.

In one embodiment, R₂₂ is methyl.

In one embodiment, R₂₂ is ethyl.

In one embodiment, R₂₃, R₂₄, and R₂₅ are methyl.

In one embodiment of Formula II:
M is —CH₂CH₂—;
X₁ is —NHC(O)—;
R₂₁ is a C₁₆-C₁₈ straight chain alkyl or C₁₆-C₁₈ straight chain alkenyl containing not more than one double bond;
R₂₂ is hydrogen, methyl, or ethyl; and
R₂₃, R₂₄, and R₂₅ are each independently hydrogen or methyl.

In one embodiment of Formula II:
M is —CH₂CH₂—;
X₁ is —NHC(O)—;
R₂₁ is a C₁₆-C₁₈ straight chain alkyl or C₁₆-C₁₈ straight chain alkenyl containing not more than one double bond;
R₂₂ is hydrogen, methyl, or ethyl; and
R₂₃, R₂₄, and R₂₅ are each methyl.

In one embodiment of Formula II,
M is —CH₂CH₂—;
X₁ is —NHC(O)—;
R₂₁ is —C₁₁H₂₃ or —C₉H₁₉;
R₂₂ is —C₂H₅ or —C₁₀H₂₁; and
R₂₃, R₂₄ and R₂₅ are each methyl.

### 5.2.3 COMPOUNDS OF FORMULA III

The compounds used in the methods of the invention also include compounds, of Formula III: 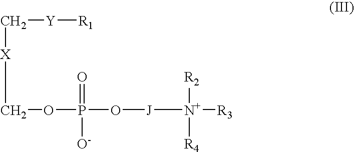
and pharmaceutically acceptable salts or prodrugs thereof,
wherein:
Y is —S—, —O—, —NH—, —N(CH₃)—, —NHC(O)—, or —N(CH₃)C(O)—;
R₁ is C₁-C₁₈ alkyl, C₂-C₁₈ alkenyl, C₂-C₁₈ alkynyl, or aryl;
X is a covalent bond or methylene that is optionally substituted with a hydroxyl, C₁-C₂₀ alkyl, —O—(C₁-C₂₀ alkyl), —S—(C₁-C₂₀ alkyl), —C(O)N(C₂-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl), or —C(O)N(C₂-C₂₀ alkynyl);
J is a C₁-C₄ alkyl that is optionally substituted one to three times with methyl or ethyl; and
R₂, R₃, and R₄ are independently hydrogen or C₁-C₃ alkyl.

In one embodiment, Y is —S—.

In one embodiment, Y is —O—.

In one embodiment, Y is —NH—.

In one embodiment, Y is —N(CH₃)—.

In one embodiment, Y is —NHC(O)—.

In one embodiment, Y is —N(CH₃)C(O)—.

In one embodiment, R₁ is C₁-C₁₈ alkyl.

In one embodiment, R₁ is C₂-C₁₈ alkenyl.

In one embodiment, R₁ is C₂-C₁₈ alkynyl.

In one embodiment, R₁ is C₁₄-C₁₈ alkyl.

In one embodiment, R₁ is C₁₄-C₁₈ alkenyl.

In one embodiment, R₁ is C₁₄-C₁₈ alkynyl.

In one embodiment, R₁ is aryl.

In one embodiment, X is a covalent bond.

In one embodiment, X is a methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl), or —C(O)N(C₂-C₂₀ alkynyl).

In one embodiment, X is a methylene that is optionally substituted with a hydroxyl, C₁-C₅ alkyl, —O—(C₁-C₅ alkyl), —S—(C₁-C₅ alkyl), —C(O)N(C₁-C₅ alkyl), C₂-C₅ alkenyl, —O—(C₂-C₅ alkenyl), —S—(C₂-C₅ alkenyl), —C(O)N(C₂-C₅ alkenyl), C₂-C₅ alkynyl, —O—(C₂-C₅ alkynyl), —S—(C₂-C₅ alkynyl), or —C(O)N(C₂-C₅ alkynyl).

In one embodiment R₂, R₃, and R₄ are methyl.

In one embodiment, J is —CH₂CH₂—.

In one embodiment of Formula III,
Y is —NHC(O)—;
R₁ is —C₁₁H₂₃ or —C₉H₁₉;
X is —CH(OC₂H₅) or —CH(OC₁₀H₂₁);
J is —CH₂CH₂—; and
R₂, R₃, and R₄ are each methyl.

### 5.2.4 COMPOUNDS OF FORMULA IV

The compounds used in the methods of the invention also include compounds of Fonmula IV: 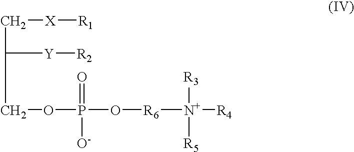
and pharmaceutically acceptable salts or prodrugs thereof,
wherein:
R₁ is a C₆-C₁₈ alkyl, C₆-C₁₈ alkenyl , or C₆-C₁₈ alkynyl that is optionally substituted from 1 to 5 times with —OH, —COOH, oxo, amino, or aryl;

X is s-NHC(O)—, —N(CH₃)C(O)—, —C(O)NH—, —C(O)N(CH₃)—, —S—, —S(O)—, —(SO₂)—, —O—, —NH—, or —N(CH₃)—;
R₂ is a C₁-C₁₄ alkyl, C₂-C₁₄ alkenyl, or C₂-C₁₄ alkynyl that is optionally substituted from 1 to 5 times with —OH, —COOH, oxo, amino, or aryl;
Y is —NHC(O)—, —N(CH₃)C(O)—, —C(O)NH—, —C(O)N(CH₃)—, —S—, —S(O)—, —(SO₂)—, —O—, —NH—, —N(CH₃)—, or —OC(O)—;
R₆ is a C₂-C₆ alkyl, C₂-C₆ alkenyl, or C₂-C₆ alkynyl; and
R₃, R₄, and R₅ are independently methyl or ethyl, or R₃ and R₄ together form an aliphatic or heterocyclic ring having five or six ring atoms and R₅ is methyl or ethyl.

In one embodiment, R₁ is a C₆-C₁₈ alkyl that is optionally substituted from 1 to 5 times with —OH, —COOH, oxo, amino.

In one embodiment, R₁ is a C₆ to C₁₈ alkenyl that is optionally substituted from 1 to 5 times with —OH, —COOH, oxo, amino.

In one embodiment, R₁ is a C₆-C₁₈ alkynyl that is optionally substituted from 1 to 5 times with —OH, —COOH, oxo, amino.

In one embodiment, R₁ is aryl.

In one embodiment, X is —NHC(O)—.

In one embodiment, X is —N(CH₃)C(O)—.

In one embodiment, X is —C(O)NH—.

In one embodiment, X is —C(O)N(CH₃)—.

In one embodiment, X is —S—.

In one embodiment, X is —S(O)—.

In one embodiment, X is —(SO₂)—.

In one embodiment, X is —O—.

In one embodiment, X is —NH—.

In one embodiment, X is —N(CH₃)—.

In one embodiment, R₂ is a C₁-C₁₄ alkyl that is optionally substituted from 1 to 5 times with —OH, —COOH, oxo, amino.

In one embodiment, R₂ is a C₂-C₁₄ alkenyl that is optionally substituted from 1 to 5 times with —OH, —COOH, oxo, amino.

In one embodiment, R₂ is a C₂-C₁₄ alkynyl that is optionally substituted from 1 to 5 times with —OH, —COOH, oxo, amino.

In one embodiment, R₂ is a C₁-C₅ alkyl that is optionally substituted from 1 to 5 times with —OH, —COOH, oxo, amino.

In one embodiment, R₂ is a C₂-C₅ alkenyl that is optionally substituted from 1 to 5 times with —OH, —COOH, oxo, amino.

In one embodiment, R₂ is a C₂-C₅ alkynyl that is optionally substituted from 1 to 5 times with —OH, —COOH, oxo, amino.

In one embodiment, R₂ is aryl.

In one embodiment, Y is —NHC(O)—.

In one embodiment, Y is —N(CH₃)C(O)—.

In one embodiment, Y is —C(O)NH—.

In one embodiment, Y is —C(O)N(CH₃)—.

In one embodiment, Y is —S—.

In one embodiment, Y is —S(O)—.

In one embodiment, Y is —(SO₂)—.

In one embodiment, Y is —O—.

In one embodiment, Y is —NH—.

In one embodiment, Y is —N(CH₃)—.

In one embodiment, Y is —OC(O)—.

In one embodiment, R₆ is a C₂-C₆ alkyl.

In one embodiment, R₆ is a C₂-C₆ alkenyl.

In one embodiment, R₆ is a C₂-C₆ alkynyl.

In one embodiment, R₃, R₄, and R₅ are methyl.

In one embodiment, R₃, R₄, and R₅ are ethyl.

In one embodiment, R₃ and R₄, together form an aliphatic or heterocyclic ring having five or six ring atoms and R₅ is methyl or ethyl.

In one embodiment of Formula IV,
X is —NHC(O)—;
R₁ is —C₁₁C₂₃ or —C₉H₁₉;
Y is —O—;
R₂ is —C₂H₅ or —C₁₀H₂₁;
R₆ is —CH₂CH₂—; and
R₃, R₄, and R₅ are each methyl.

Exemplary compounds of formula I-IV useful in the methods of the invention include, but are not limited to, 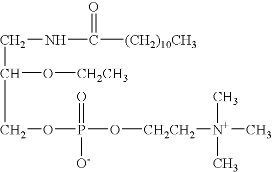
3-dodecanamido-2-ethoxypropyl-1-phosphocholine, 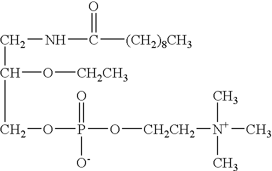
3-decanamido-2-ethoxypropyl-1-phosphocholine, 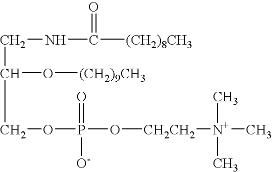
3-decanamido-2-decyloxypropyl-1-phosphocholine, 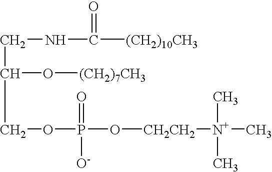
3-dodecanamido-2-octyloxypropyl-1-phosphocholine, 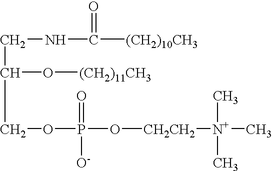
3-dodecanamido-2-dodecyloxypropyl-1-phosphocholine, or 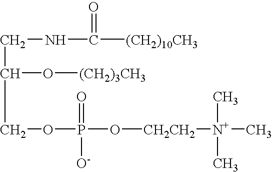
3-dodecanamido-2-butyloxy-1-phosphocholine; or a combination thereof.

### 5.2.5 COMPOUNDS OF FORMULA AA

The compounds used in the methods of the invention also include compounds of Formula AA: 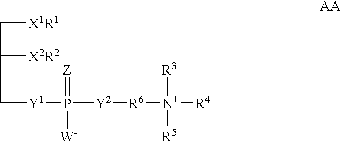
and pharmaceutically acceptable salts or prodrugs thereof,
wherein:
R¹ is an alkyl, alkenyl, or alkynyl that is optionally substituted from 1 to 5 times with —OH, —COOH, oxo, or amino;
R² is an alkyl, alkenyl, or alkynyl that is optionally substituted from 1 to 5 times with —OH, —SH, oxo, amino, —N(R′)C(O)R″, —C(O)N(R′)(R″), —COOH, or —COOR′;
X¹ and X² are each selected independently from the group consisting of —N(R′)—N(R″)—, —NHC(O)—, —N(R′)C(O)—, —N(CH₃)C(O)—, —C(O)NH—, —C(O)N(R′)—, —C(O)N(CH₃)—, —NH—, —N(R′)—, —N(CH₃)—, —(C═NH)—, —(C═NR′)—, —O(C═NH)—, —O(C═NR′)—, —(C═NH)O—, —(C═NR′)O—, —S(C═NH)—, —S(C═NR′)—, —(C═NH)S—, —(C═NR′)S—, —O(C═NH)O—, —S(C═NH)O—, —O(C═NH)S—, —S(C═NH)S—, —O(C═NR′)O—, —S(C═NR′)O—, —O(C═NR′)S—, —S(C═NR′)S—, —C(O)—, —OC(O)—, —C(O)O—, —OC(O)O—, —SC(O)—, —C(O)S—, —SC(O)O—, —OC(O)S—, —SC(O)S—, —NHC(O)NH—, —NHC(O)N(R′)—, —N(R′)C(O)NH—, —N(R′)C(O)N(R″)—, —NHC(S)—, —NR′C(S)—, —N(CH₃)C(S)—, —C(S)NH—, —C(S)N(R′)—, —C(S)N(CH₃)—, —C(S)—, —OC(S)—, —C(S)O—, —OC(S)O—, —SC(S)—, —C(S)S—, —SC(S)O—, —OC(O)S—, —SC(S)S—, —NHC(S)NH—, —NHC(S)N(R′)—, —N(R′)C(S)NH—, —N(R′)C(S)N(R″)—, —O—, —S—, —S(O)—, —(SO₂);
Y¹ and Y² are selected independently from the group consisting of —O—, —S— and —Se—;
Z is —O—, —S—, —Se—, —NH—, or —N(R′)—;
W is —O—, —S—, —NH—, or —N(R′)—;
R⁶ is alkyl, alkenyl, or alkynyl.
R³, R⁴, and R⁵ are each independently an alkyl group, or R³ and R⁴ together form a heterocyclic ring having between three and seven ring atoms and R⁵ is an alkyl group; and
R′ and R″ are each independently hydrogen, alkyl, alkenyl, alkynyl, cycloalkyl, aryl, heteroaryl, or heterocylic.

In one embodiment, X¹ is —NHC(O)—.

In one embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—

In one embodiment, X¹ is —N(CH₃)—.

In one embodiment, X² is —NHC(O)—.

In one embodiment, X² is —N(CH3)C(O)—.

In one embodiment, X² is —C(O)_{N}(CH₃)—.

In one embodiment, X² is —S—.

In one embodiment, X² is —S(O)—.

In one embodiment, X² is —(SO₂)—.

In one embodiment, X² is —O—.

In one embodiment, X² is —NH—.

In one embodiment, X² is —N(CH₃)—.

In one embodiment, Y¹ is —(O)—.

In one embodiment, Y¹ is —S—.

In one embodiment, Y¹ is —Se—.

In one embodiment, Y² is —O—.

In one embodiment, Y² is —S—.

In one embodiment, Y² is —Se—.

In one embodiment, Z is —O—.

In one embodiment, Z is —S—.

In one embodiment, Z is —Se—.

In one embodiment, Z is —NH—.

In one embodiment, Z is —NR—.

In one embodiment, W is —O—.

In one embodiment, W is —S—.

In one embodiment, W is —NH—.

In one embodiment, W is —NR′—.

In one embodiment, R¹ is a C₁-C₂₂ alkyl optionally substituted from 1 to 5 times with —OH, —COOH, oxo, or amino.

In one embodiment, R¹ is a C₁-C₁₂ alkyl optionally substituted from 1 to 5 times with —OH, —COOH, oxo, or amino.

In one embodiment, R¹ a C₂-C₂₂ alkenyl optionally substituted from 1 to 5 times with —OH, —COOH, oxo, or amino.

In one embodiment, R¹ is a C₂-C₁₂ alkenyl optionally substituted from 1 to 5 times with —OH, —COOH, oxo, or amino.

In one embodiment, R¹ a C₂-C₂₂ alkynyl optionally substituted from 1 to 5 times with —OH, —COOH, oxo, or amino.

In one embodiment, R¹ is a C₂-C₁₂ alkynyl optionally substituted from 1 to 5 times with —OH, —COOH, oxo, or amino.

In one embodiment, R² a C₁-C₂₂ alkyl optionally substituted from 1 to 5 times with —OH, —SH, oxo, amino, —N(R′)C(O)R″, —C(O)N(R′)(R″), —COOH, or —COOR′.

In one embodiment, R² is a C₁-C₁₂ alkyl optionally substituted from 1 to 5 times with —OH, —SH, oxo, amino, —N(R′)C(O)R″, —C(O)N(R′)(R″), —COOH, or —COOR′.

In one embodiment, R² a C₁-C₅ alkyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₂₂ alkenyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₁₂ alkenyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₅ alkenyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₂₂ alkynyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₁₂ alkynyl optionally substituted from 1 to 5 times with —OH, —SH, oxo, amino, —N(R′)C(O)R″, —C(O)N(R′)(R″), —COOH, or —COOR′.

In one embodiment, R² is a C₂-C₅ alkynyl optionally substituted from 1 to 5 times with —OH, —SH, oxo, amino, —N(R′)C(O)R″, —C(O)N(R′)(R″), —COOH, or —COOR′.

In one embodiment, R⁶ is a C₂-C₆ alkyl.

In one embodiment, R⁶ is —CH₂—.

In one embodiment, R⁶ is —CH₂—CH₂—.

In one embodiment, R⁶ is a C₂-C₆ alkenyl.

In one embodiment, R⁶ is a C₂-C₆ alkynyl.

In one embodiment, R³, R⁴, and R⁵ are each independently a C₁-C₆ alkyl.

In one embodiment, each R³, R⁴ and R⁵ is independently a methyl or ethyl.

In one embodiment, R³ and R⁴ together form a heterocyclic ring having between three and seven ring atoms and R⁵ is an alkyl group.

In one embodiment, R³ and R⁴ together form a heterocyclic ring having between three and seven ring atoms and R⁵ is methyl.

In one embodiment, R³ and R⁴ together form a heterocyclic ring having between three and seven ring atoms and R⁵ is ethyl.

In one embodiment, each R′ and R″ is independently a C₁-C₂₂ alkyl group.

In one embodiment, compounds useful in the methods of the invention include compounds of Formula AA-1: 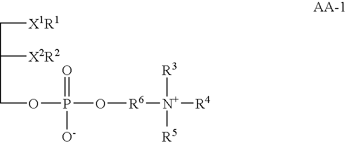
and pharmaceutically acceptable salts or prodrugs thereof,
wherein:
R¹ is an C₁-C₁₂ alkyl, C₂-C₁₂ alkenyl, or C₂-C₁₂ alkynyl that is optionally substituted from 1 to 5 times with —OH, —COOH, oxo, or amino;
X¹ is —NHC(O)—, —N(CH₃)C(O)—, —C(O)NH—, —C(O)N(CH₃)—, —NH— or —N(CH₃)—;
R² is C₁-C₁₂ alkyl, C₂-C₁₂ alkenyl, or C₂-C₁₂ alkynyl that is optionally substituted from 1 to 5 times with —OH, —COOH, oxo, or amino;
X² is —NHC(O)—, —N(CH₃)C(O)—, —C(O)N(CH₃)—, —S—, —S(O)—, —(SO₂)—, —O—, —NH—, or —N(CH₃)—;
R⁶ is C₁-C₆ alkyl, C₂-C₆ alkenyl, C₂-C₆ alkynyl; and
R³, R⁴, and R⁵ are each independently methyl or ethyl, or R³ and R⁴ together form a heterocyclic ring having five or six ring atoms and R⁵ is methyl or ethyl.

In one embodiment, R¹ is a C₁-C₁₂ alkyl substituted from 1 to 5 times with —OH, —COOH, oxo, or amino.

In one embodiment, R¹ is a C₂-C₁₂ alkenyl substituted from 1 to 5 times with —OH, —COOH, oxo, or amino.

In one embodiment, R¹ is a C₂-C₁₂ alkynyl substituted from 1 to 5 times with —OH, —COOH, oxo, or amino.

In one embodiment, R¹ is a C₁-C₅ alkyl substituted from 1 to 5 times with —OH, —COOH, oxo, or amino.

In one embodiment, R¹ is a C₂-C₅ alkenyl substituted from 1 to 5 times with —OH, —COOH, oxo, or amino.

In one embodiment, R¹ is a C₂-C₅ alkynyl substituted from 1 to 5 times with —OH, —COOH, oxo, or amino.

In one embodiment, X¹ is —NHC(O)—.

In one embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—.

In one embodiment, X¹ is —N(CH₃)—.

In one embodiment, R² is a C₁-C₁₂ alkyl that is optionally substituted from 1 to 5 times with —OH, —COOH, oxo, or amino.

In one embodiment, R is a C₂-C₁₂ alkenyl that is optionally substituted from 1 to 5 times with —OH, —COOH, oxo, or amino.

In one embodiment, R² is a C₂-C₁₂ alkynyl that is optionally substituted from 1 to 5 times with —OH, —COOH, oxo, or amino.

In one embodiment, R is a C₁-C₅ alkyl substituted from 1 to 5 times with —OH, —COOH, oxo, or amino.

In one embodiment, R is a C₂-C₅ alkenyl substituted from 1 to 5 times with —OH, —COOH, oxo, or amino.

In one embodiment, R is a C₂-C₅ alkynyl substituted from 1 to 5 times with —OH, —COOH, oxo, or amino.

In one embodiment, X² is —NHC(O)—.

In one embodiment, X² is —N(CH₃)C(O)—

In one embodiment, X² is —C(O)N(CH₃)—.

In one embodiment, X² is —S—.

In one embodiment, X² is —S(O)—.

In one embodiment, X² is —(SO₂)—.

In one embodiment, X² is —O—.

In one embodiment, X²is —NH—.

In one embodiment, X² is —N(CH₃)—.

In one embodiment, R⁶ is a C₂-C₆ alkyl.

In one embodiment, R⁶ is a C₂-C₆ alkenyl.

In one embodiment, R⁶ is a C₂-C₆ alkynyl.

In one embodiment, each R³, R⁴, and R⁵ is independently methyl or ethyl.

In one embodiment, R³ and R⁴ together form a heterocyclic ring having five or six ring atoms and R⁵ is methyl.

In one embodiment, R³ and R⁴ together form a heterocyclic ring having five or six ring atoms and R⁵ is ethyl.

In one embodiment, compounds useful in the methods of the invention include compounds of Formula AA-1: 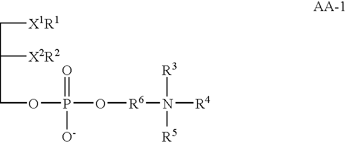
and pharmaceutically acceptable salts or prodrugs thereof,
wherein:
X¹ is —NHC(O)—, —N(CH₃)C(O)—, —C(O)NH—, —C(O)N(CH₃)—, —NH— or —N(CH₃)—;
X² is —NHC(O)—, —N(CH₃)C(O)—, —C(O)N(CH₃)—, —S—, —S(O)—, —(SO₂)—, —O—, —NH—, or —N(CH₃)—;
R¹ is an C₁-C₂₂ alkyl, C₂-C₂₂ alkenyl, or C₂-C₂₂ alkynyl;
R² is an C₁-C₁₂ alkyl, C₂-C₁₂ alkenyl, or C₂-C₁₂ alkynyl;
   and at least one of R¹ or R² is a C₁-C₇ alkyl, C₂-C₇ alkenyl, or C₂-C₇ alkynyl;
R⁶ is a C₁-C₆ alkyl, C₂-C₆ alkenyl, or C₂-C₆ alkynyl; and
R³, R⁴, and R⁵ are each independently methyl or ethyl or R³ and R⁴ together form a heterocyclic ring having five or six ring atoms and R⁵ is methyl or ethyl.

In one embodiment, one or more alkyl groups is substituted.

In one embodiment, X¹ is —NHC(O)—.

In one embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—.

In one embodiment, X¹ is —N(CH₃)—.

In one embodiment, X² is —NHC(O)—.

In one embodiment, X² is —N(CH₃)C(O)—

In one embodiment, X² is —C(O)N(CH₃)—.

In one embodiment, X² is —S—.

In one embodiment, X² is —S(O)N.

In one embodiment, X² is —(SO₂)—.

In one embodiment, X² is —O—.

In one embodiment, X² is —NH—.

In one embodiment, X² is —(NCH₃)—

In one embodiment, R¹ is a C₁-C₁₂ alkyl.

In one embodiment, R¹ is a C₂-C₁₂ alkenyl.

In one embodiment, R¹ is a C₂-C₁₂ alkynyl.

In one embodiment, R² is a C₁-C12 alky

In one embodiment, R² is a C₂-C₁₂ alkenyl.

In one embodiment, R² is a C₂-C₁₂ alkynyl.

In one embodiment, at least one of R¹ or R² is a C₁-C₃ alkyl, C₂-C₃ alkenyl, or C₂-C₃ alkynyl.

In one embodiment, at least one of R¹ or R² is a C₁-C₃ alkyl.

In one embodiment, at least one of R¹ or R² is a C₂-C₃ alkenyl.

In one embodiment, at least one of R¹ or R² is a C₂-C₃ alkynyl group.

In one embodiment, R² is a C₁-C₅ alkyl.

In one embodiment, R² is a C₂-C₅ alkenyl.

In one embodiment, R² is a C₂-C₅ alkynyl group.

In one embodiment, R⁶ is a C₂-C₆ alkyl.

In one embodiment, R⁶ is a C₂-C₆ alkenyl.

In one embodiment, R⁶ is a C₂-C₆ alkynyl.

In one embodiment of the compound of formula AA-1,
X¹ is —NHC(O)—;
X² is —S— or —O—;
R¹ and R² are each a CC₁-C₂₂ straight chain alkyl and at least one of R¹ or R²is a C₁-C₅ straight chain alkyl;
R⁶ is a C₂-C₆ straight chain alkyl; and
R³, R⁴, and R⁵ are each independently methyl or ethyl.

In one embodiment of the compound of formula AA-1,
X¹ is —NHC(O)—;
X² is —S— or —O—;
R¹ and R² are each a C₁-C₁₂ straight chain alkyl and at least one of R¹ or R²is a C₁-C₅ straight chain alkyl;
R⁶ is a C₂-C₆ straight chain alkyl; and
R³, R⁴, and R⁵ are each independently methyl or ethyl.

In one embodiment of the compound of formula AA-1,
X¹ is —NHC(O)—;
X² is —S— or —O—;
R¹ is a C₁-C₂₂ straight chain alkyl;
R² is a C₁-C₅ straight chain alkyl;
R⁶ is —CH₂CH₂—; and
R³, R⁴, and R⁵ are each methyl.

In one embodiment of the compound of formula AA-1,
X¹ is —NHC(O)—;
X² is —S—or —O—;
R¹ is a C₇-C₁₁ straight chain alkyl;
R² is a C₁-C₅ straight chain alkyl;
R⁶ is a —CH₂CH₂—; and
R³, R⁴, and R⁵ are each methyl.

In one embodiment of the compound of formula AA-1,
X¹ is —NHC(O)—;
X² is —S—or —O—;
R¹ is a C₇-C₁₁ straight chain alkyl;
R² is a methyl or ethyl;
R⁶ is a —CH₂CH₂—; and
R³, R⁴, and R⁵ are each methyl.

In one embodiment of the compound of formula AA-1,
X¹ is —NHC(O)—;
X² is —O—;
R¹ is —C₁-C₂₂ alkyl;
R² is —C₁-C₂₂ alkyl;
R⁶ is —CH₂CH₂—; and
R³, R⁴ and R⁵ are methyl.

In one embodiment of the compound of formula AA-1,
X¹ is —NHC(O)—;
X² is —O—;
R¹ is —CH₃, —CH₂CH₃, —CH₂CH₂CH₃, —CH₂CH₂CH₂CH₃, —CH₂CH₂CH₂Ch₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃;
R² is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀Ch₃, —(CH₂)₁₁CH₃, —(CH₂)₁₂CH₃ or —(CH₂)₁₃CH₃;
R⁶ is —CH₂CH₂—; and
R³, R⁴ and R⁵ are methyl.

In one embodiment of the compound of Formula AA-1,
X¹ is —NHC(O)—;
X² is —O—;
R¹ ^{_{—(CH}}₂)₈CH₃, —(CH₂)₉CH₃, —(CH₂)₁₀CH₃, —(CH₂)₁₁CH₃; —(CH₂)₁₂CH₃, or —(CH₂)₁₃CH₃;
R²is CH₃, —CH₂CH₃, —CH₂CH₂CH₃, —CH₂CH₂CH₂CH₃, —CH₂CH₂Ch₂CH₂CH₃, —(CH₂)₅CH₃, —(CH₂)₆CH₃, or —(CH₂)₇CH₃;
R⁶ is —CH₂CH₂—; and
R³, R⁴ and R⁵ are methyl.

In one embodiment of the compound of Formula AA-1,
X¹ is —NHC(O)—;
X² is —O—;
R¹ is —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, or —(CH₂)₁₂CH₃;
R² is —(CH₂)₆CH₃, —(CH₂)₇CH₃, —(CH₂)₈CH₃, —(CH₂)₉CH₃, —(CH₂)₁CH₃, —(CH₂)₁₁CH₃, —(CH₂)₁₂CH₃, or —(CH₂)₁₃CH₃;
R⁶ is —CH₂CH₂—; and
R³, R⁴ and R⁵ are methyl.

### 5.2.6 COMPOUNDS OF FORMULA BB

The compounds used in the methods of the invention also include compounds of Formula BB-1: 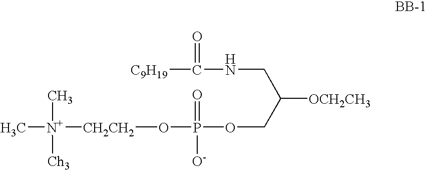
and pharmaceutically acceptable salts or prodrugs thereof.

The compounds used in the methods of the invention also include compounds of Formula BB-2: 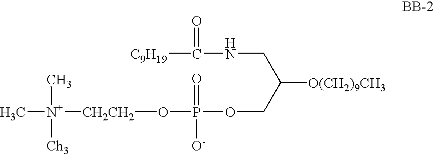
and pharmaceutically acceptable salts or prodrugs thereof.

The compounds used in the methods of the invention also include compounds of Formula BB-3: 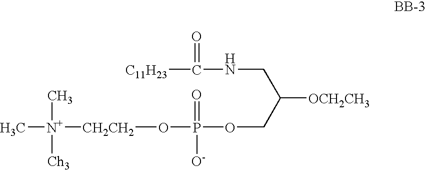
and pharmaceutically acceptable salts or prodrugs thereof.

The compounds used in the methods of the invention also include compounds of Formula BB-4: 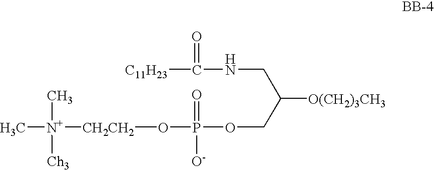
and pharmaceutically acceptable salts or prodrugs thereof.

### 5.2.7 OTHER COMPOUNDS USEFUL IN THE METHODS OF THE INVENTION

The compounds used in the methods of the invention also include the phospholipids compounds disclosed in PCT publication WO 91/09602 (Boehringer Mannheim), which is incorporated herein in its entirety, and in particular phospholipids of the formula: 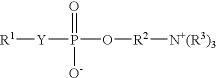
and pharmaceutically acceptable salts or prodrugs thereof,
wherein:
R¹ is a straight-chain or branched, saturated or unsaturated aliphatic residue, in particular an alkyl residue, with 9 to 30 carbon atoms, which can also be part of a C₅-C₇ cycloalkane ring and may be substituted with one or more hydroxy, halogen, nitrile, a C₅-C₇ cycloalkyl, phenyl, C₁-C₂₀ alkoxy carbonyl, C₁-C₂₀ alkyl carbonyl, C₁-C₂₀ alkyl carbamoyl, C₁-C₂₀ alkyl mercapto, C₁-C₂₀ alkane sulphinyl, C₁-C₂₀ alkane sulphonyl, C₁-C₂₀ acyl amino groups or by C₁-C₂₀ alkoxy which in turn can be substituted by phenyl, C₁-C₂₀ alkyl mercapto, C₁-C₂₀ alkane sulphinyl, C₁-C₂₀ alkane sulphonyl, C₁-C₂₀ acyl amino, C₁-C₂₀ alkoxy carbonyl, nitrile, hydroxy, C₁-C₂₀ alkoxy or C₁-C₂₀ alkyl carbamoyl;
R² is a straight-chain or branched alkylene chain with 2 to 6, preferably 2 to 4, carbon atoms;
R³ is hydrogen or a C₁-C₆ alkyl group; and

Y is an oxygen or a sulphur atom.

The compounds used in the methods of the invention also include the phospholipid compounds disclosed in WO 91/05558 (Boehringer Mannheim), which is incorporated herein in its entirety, and in particular phospholipids of the formula: 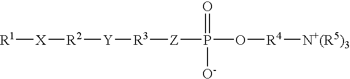
and pharmaceutically acceptable salts or prodrug thereof,
wherein:
X is a valence bond, an oxygen atom or sulphur atom, a sulphinyl, sulphonyl, carbonyl, aminocarbonyl, carbonylamino or ureido (—NH—CO—NH—) group or a C₃-C₈ cycloalkylene or phenylene residue;
Y is an oxygen atom or the groups —O—CO—O—, —O—CO—NH—, —O—CS—NH—;
R¹ is a hydrogen atom, a straight-chain or branched, saturated or unsaturated alkyl residue with 1-18 or 2-18 carbon atoms, respectively, which may be substituted one or more times by phenyl, halogen, C₁-C₄ alkoxy, C₁-C₄ alkylmercapto, C₁-C₄ alkoxycarbonyl, C₁-C₄ alkane sulphinyl or C₁-C₄ alkane sulphonyl groups,
R² is a straight or branched, saturated or unsaturated alkylene chain with 1-18 or 2-18 carbon atoms, respectively, which may be substituted one or more times by halogen, phenyl, C₁-C₄ alkoxy, C₁-C₄ alkoxycarbonyl, C₁-C₄ alkylmercapto, C₁-C₄ alkane sulphinyl or C₁-C₄ alkane sulphonyl groups;
R³is a straight or branched, saturated or unsaturated alkylene chain with 2-8 carbon atoms which can also be substituted;
R⁴is a straight or branched alkylene chain with 2-5 carbon atoms;
R⁵ is hydrogen or a C₁-C₆ alkyl group; and
Z is oxygen or sulphur.

The compounds used in the methods of the invention also include the phospholipid compounds disclosed in U.S. Pat. No. 4,444,766 (Boehringer Mannheim), which is incorporated herein in its entirety, and in particular phospholipids of the formula: 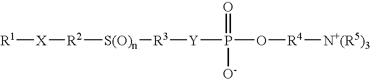
and pharmaceutically acceptable salts or prodrug thereof,
wherein:
X is a valency bond, an oxygen or sulphur atom, a sulphonyl or sulphonyl group, an aminocarbonyl, carbonylamino or ureido group or a cycloalkylene radical or a phenylene radical;
Y is an oxygen or sulphur atom;
R¹ is a hydrogen atom, a straight-chained or branched, saturated or unsaturated aliphatic hydrocarbon radical containing up to 18 carbon atoms, which is optionally substituted one or more times by aryl, halogen, lower alkoxy, alkylthio, alkoxycarbonyl, alkanesulphinyl or alkanesulphonyl;
R² is a straight-chained or branched, saturated or unsaturated aliphatic hydrocarbon chain containing up to 18 carbon atoms, which is optionally substituted one or more times by halogen, aryl, lower alkoxy, alkoxycarbonyl, alkylthio, alkanesulphinyl or alkanesulphonyl,
R³ is a straight-chained or branched, saturated or unsaturated aliphatic hydrocarbon chain containing 2 to 8 carbon atoms, which can also be part of a cycloalkane ring and which is optionally substituted one or more times by hydroxy, halogen, nitrile, cycloalkyl, phenyl, alkoxycarbonyl, optionally alkylated carbamoyl, alkylthio, alkanesulphinyl, alkanesulphonyl, optionally acylated amino or by alkoxy which, in turn, can be substituted by aryl, alkylthio, alkanesulphinyl, alkanesulphonyl, optionally acylated amino, alkoxycarbonyl, nitrile, hydroxyl, alkoxy or optionally alkylated carbamoyl;
R⁴ is a straight-chained or branched alkylene chain containing 2 to 4 carbon atoms;
R⁵ is a hydrogen atom or a lower,alkyl radical;
and n is 0, 1 or 2

In addition, exemplary compounds include any of the compounds disclosed in Ouyang et al., Journal of Medicinal Chemistry 45:2857-2866 (2002), the disclosure of which is hereby incorporated by reference. Other compounds that may be used, alone or in combination, for the treatment of RSV, as disclosed herein, include compounds disclosed in U.S. Pat. No. 5,614,548, U.S. Pat. No. 5,962,437, and U.S. Pat. No. 5,770,584, the disclosures of which are hereby incorporated by reference.

The invention also provides the use of a compound of the invention, optionally with a pharmaceutical acceptable carrier or diluent, for the manufacture of a medicament for the treatment of a host infected with RSV.

### 5.3 METHODS OF SYNTHESIS OF COMPOUNDS

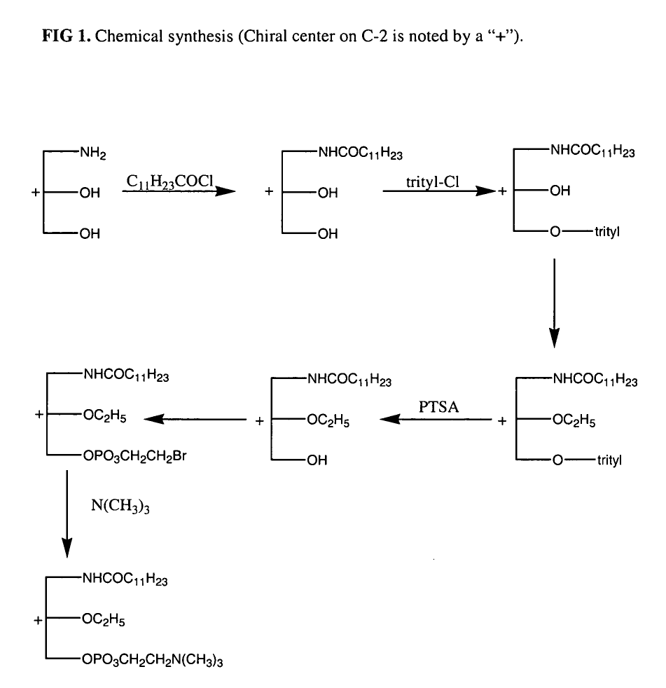

Methods for obtaining the compounds of the invention are well known to those skilled in the art. Alkylamidophosphocholines may be prepared according to the method disclosed in Ouyang et al., Journal of Medicinal Chemistry 45(13): 2857-2866 (2002), and as described in Example 3 and FIG. 1. 3-Alkylamido-2-alkoxypropylphosphocholine is obtained by reacting commercially available 3-amino-1,2-propanediol with the appropriate acid chloride or anhydride. The primary alcohol is protected, and the secondary alcohol is alkylated with an alkyl bromide. The primary alcohol is deprotected, and then reacted with 2-bromoethyl dichiorophosphate and trimethylamine, to obtain the 3-alkylamido-2-alkoxypropylphosphocholine compound.

The compounds of the invention can also be prepared using the methods disclosed in Morris-Natschke S L, Gumus F, Marasco C J, Meyer K L, Marx M, Piantadosi, Layne M D, Modest E J, “Synthesis of Phosphocholine and Quaternary Amine Ether Lipids and Evaluation of In Vitro Antineoplastic Activity,” Journal of Medicinal Chemistry, 36:2018-2025 (1993); Piantadosi C., Marasco C. J., Morris-Natschke S. L., Meyer K. L., Gumus F., Surles J. R., Ishaq K. S., ” Synthesis and Evaluation of Novel Ether Lipid Nucleoside Conjugates for Anti-HIV-1 Activity,” Journal of Medicinal Chemistry, 34:1408-1414 (1991); Kucera L. S., Morris-Natschke S. L., Ishaq K. S., Hes J., Iyer N., Furman P. A., Fleming R. A., ” Synthesis and Evaluation of a Novel Synthetic Phosphocholine Lipid-AZT Conjugate that Double-Targets Wild-type and Drug Resistant Variants of HIV,” Nucleosides, Nucleotides, and Nucleic Acids, 23:385-399 (2004); Meyer K. L., Marasco C. J., Morris-Natschke S. L., Ishaq K. S., Piantadosi C., Kucera L. S.,” In Vitro Evaluation of Phosphocholine and Quaternary Ammonium Containing Lipids as Novel Anti-HIV Agents,” Journal of Medicinal Chemistry, 34:1377-1383 (1991); and Morris-Natschke S. L., Surles J. R., Daniel L. W., Berens M. E., Modest E. J., Piantadosi C., ” Synthesis of Sulfur analogues of Alkyl Lysophospholipid and Neoplastic Cell Growth Inhibitory Properties,” Journal of Medicinal Chemistry, 29:2114-2117 (1986).

Other compounds which may be used, alone or in combination, for the treatment of RSV, as disclosed herein, and the synthesis thereof, are disclosed in U.S. Pat. No. 5,614,548, U.S. Pat. No. 5,962,437, and U.S. Pat. No. 5,770,584, which are hereby incorporated by reference.

### 5.4 PREPARATION AND ADMINISTRATION OF COMPOUNDS

The compounds of the invention may be prepared in the form of a pharmaceutically acceptable salt or a non-pharmaceutically acceptable salt. Non-pharmaceutically acceptable salts are useful, for example, as intermediates for preparation of a pharmaceutically acceptable salt. When the compounds are sufficiently basic or acidic to form stable non-toxic acid or base salts, the compounds may be prepared as a pharmaceutically acceptable salt. Pharmaceutically acceptable salts are salts that retain the desired biological activity of the parent compound and do not impart undesirable toxicological effects.

Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

The compounds of the invention may be formulated as pharmaceutical compositions and administered to a host, such as a human patient, by a chosen route of administration. Pharmaceutical compositions that are useful in the methods of the invention can be prepared, packaged, or sold in a variety of formulations which can be suitable for one or more routes of administration such as, for example, oral, intravenous, intramuscular, topical, subcutaneous, rectal, vaginal, parenteral, pulmonary, intranasal, buccal, ophthalmic, or another route of administration. The active materials can be administered in liquid or solid form. Other contemplated formulations include projected nanoparticles, liposomal preparations, resealed erythrocytes containing the active ingredient, and immunologically-based formulations.

Although the descriptions of pharmaceutical compositions provided herein are principally directed to pharmaceutical compositions which are suitable for ethical administration to humans, it will be understood by the skilled artisan that such compositions are generally suitable for administration to hosts of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates and mammals including commercially relevant mammals such as cattle, pigs, horses, sheep, cats, and dogs.

Thus, the compounds of the invention may be systemically administered (e.g. orally) in combination with a pharmaceutically acceptable vehicle such as an inert diluent or an assimilable edible carrier. They can be enclosed in hard or soft shell gelatin capsules, compressed into tablets, or incorporated directly into the food of the patient's diet. For oral therapeutic administration, the active compound can be combined with one or more excipients and used in the form of ingestible tablets, buccal tablets, troches, capsules, elixirs, suspensions, syrups, wafers, and the like.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time. Pharmaceutically compatible binding agents, and/or adjuvant materials may also be included as part of the composition.

Such compositions and preparations should contain at least 0.1% (w/w) of active compound. The percentage of the compositions and preparations can, of course, be varied, for example from about 0.1% to nearly 100% of the weight of a given unit dosage form. The amount of active compound in such therapeutically useful compositions is such that an effective dosage level will be obtained upon administration.

The tablets, froches, pills, capsules, and the like may also contain one or more of the following: binders, such as microcrystalline cellulose, gum tragacanth, acacia, corn starch, or gelatin; excipients, such as dicalcium phosphate, starch or lactose; a disintegrating agent, such as corn starch, potato starch, alginic acid, primogel, and the like; a lubricant, such as magnesium stearate or Sterotes; a glidant, such as colloidal silicon dixoide; a sweetening agent, such as sucrose, fructose, lactose, saccharin, or aspartame; a flavoring agent such as peppermint, methylsalicylate, oil of wintergreen, or cherry flavoring; and a peptide antiviral agent, such as envuvirtide (Fuzeon™). When the unit dosage form is a capsule, it can contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules can be coated with gelatin, wax, shellac, sugar, and the like. A syrup or elixir can contain the active compound, sucrose or fructose as a sweetening agent, methyl and propylparabens as preservatives, a dye, and flavoring such as cherry or orange flavor. Of course, any material used in preparing a unit dosage form should be pharmaceutically acceptable and substantially non-toxic in the amounts employed. In addition, the active compound may be incorporated into sustained-release preparations and devices.

The compounds of the invention thereof may also be mixed with other active materials that do not impair the desired action, or with materials that supplement the desired action, such as antibiotics, antifungals, antiinflammatories, protease inhibitors, or other nucleoside or nonnucleoside antiviral agents. Solutions or suspensions used for parenteral, intradermal, subcutaneous, or topical application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules, disposable syringes or multiple dose vials made of glass or plastic.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylacetic acid. Methods for preparation of such formulations will be apparent to those skilled in the art. The materials may also be obtained commercially from Alza Corporation.

The active compound may be administered orally, intravenously or intraperitoneally by infusion or injection. Solutions of the active compound or its salts may be prepared in water, optionally mixed with a non-toxic surfactant. Dispersions may be prepared in glycerol, liquid polyethylene glycols, triacetin, mixtures thereof, and in oils. Under ordinary conditions of storage and use, these preparations contain a preservative to prevent growth of microorganisms.

Pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form should be sterile, fluid, and stable under conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity may be maintained, for example, by formation of liposomes, by the maintenance of the required particle size (in the case of dispersions) or by use of one or more surfactants. Microbial growth may be prevented using various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions may be achieved using agents which delay absorption, for example, aluminum monostearate and gelatin.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

Sterile injectable solutions are prepared by incorporating the active compound in the required amount in an appropriate solvent, optionally with one or more of the other ingredients enumerated above, followed by filter sterilization. In the case of sterile powders for preparation of sterile injectable solutions, preferred methods of preparation include vacuum drying and the freeze drying techniques, which yield a powder of the active ingredient and any additional desired ingredient present in the previously-sterile-filtered solution(s).

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or a solution for vaginal irrigation.

Methods for impregnating or coating a material with a chemical composition are known in the art, and include, but are not limited to, methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations which are useful include those which comprise the active ingredient in microcrystalline form or in a liposomal preparation.

For topical administration, the present compounds can be applied in pure form, i.e., as a liquid. However, it will generally be desirable to administer the compounds to the skin as compositions or formulations, in combination with a dermatologically acceptable carrier, which may be a solid or a liquid.

Useful solid carriers include finely divided solids such as talc, clay, microcrystalline cellulose, silica, alumina, and the like. Useful liquid carriers include water, alcohols, glycols, and blends of two or more of these, in which the present compounds can be dissolved or dispersed at effective levels, optionally with the aid of non-toxic surfactants. Adjuvants such as fragrances and additional antimicrobial agents can be added to optimize properties for a given use. The resulting liquid compositions can be applied using absorbent pads, used to impregnate bandages or other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are disclosed in Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Accordingly, the invention includes pharmaceutical compositions comprising one or more compounds of the invention or any combination thereof, or a pharmaceutically acceptable salt thereof, in combination with a pharmaceutically acceptable carrier.

In one embodiment, the pharmaceutical composition is adapted for oral, topical, or parenteral administration to a mammal, such as a human, and comprises one or more compounds of the invention, or any combination thereof, or a pharmaceutically acceptable salt thereof, in an amount effective to treat RSV.

As used herein, “treatment” of RSV can mean, for example, any one or more of the following: inhibiting the replication of the virus, reducing the virus load within a patient, inhibiting formation of infectious progeny virus, inhibiting infectiousness of virus, killing cells harboring virus, interfering with one or more stages of the virus life cycle, inhibiting one or more viral enzymes or inducing production of non-infectious virus particles that can activate an immune response against infectious virus (e.g. autovaccination).

Useful dosages of the compounds of the invention for inclusion in the pharmaceutical compositions of the invention can be determined by comparing in vitro activity and in vivo activity of the compounds in appropriate animal models. Methods for the extrapolation of effective dosages in mice and other animal models to humans are known in the art (see, for example, U.S. Pat. No. 4,938,949).

Generally, the concentration of the compound(s) of the invention in a liquid composition, such as a lotion, will range from about 0.1% to about 95% by weight, preferably from about 0.5 % to about 25% by weight. The concentration in a semi-solid or solid composition such as a gel or a powder will range from about 0.1% to 100% by weight, preferably about 0.5% to about 5 % by weight. Single doses for intravenous injection, subcutaneous, intramuscular or topical administration, infusion, ingestion or suppository will generally be from about 0.001 to about 5000 mg, and be administered from about 1 to about 3 times daily, to yield levels of about 0.01 to about 500 mg/kg, for adults.

The invention also includes one or more compounds of the invention, or any combination thereof, in an amount effective to inhibit RSV replication in a host. The compound of course is therefore useful for inhibiting virus replication in a cell or neutralization (i.e. inactivation) of extracellular virus. Additionally, the invention includes one or more compound of the invention present as a pharmaceutically acceptable salt, or any combination thereof, wherein the compound is present in an amount effective to inhibit RSV replication in a host.

As used herein, to inhibit RSV replication in a host means to reduce the virus load in a host to a level which is lower than the level of the virus load in an otherwise identical mammal which was not administered the compound. Preferably, virus load in a mammal is reduced by about 1 to 12 log₁₀ or more relative to an otherwise identical mammal which was not administered the compound. Virus load in a mammal can be assessed by a number of methods known in the art such as, for example, obtaining a tissue or fluid sample from the mammal and assessing the amount of virus or viral components in the mammal contained therein using technology which is either virological, immunological, biochemical or molecular biological in nature and which is well known to the skilled artisan and which are described elsewhere herein. Inhibition of RSV replication in a cell is assessed using similar or identical assays as those used to assess virus load in a mammal.

The invention also includes a kit for administering a composition of the invention (e.g. a compound of the invention, a pharmaceutically acceptable salt thereof, or a pharmaceutical composition of the invention) to a host for treatment of RSV infection. Preferably, the host is a human. The kit comprises the composition of the invention and an instructional material, which describes adventitially administering the composition to the mammal by any of the routes of administration described herein. In another embodiment, this kit comprises a (preferably sterile) solvent suitable for dissolving or suspending the composition of the invention prior to administering the compound to the mammal.

As used herein, an ” instructional material” includes a publication, a recording, a diagram, or any other medium of expression which can be used to communicate the usefulness of the composition of the invention in the kit for any one or more of the following: effecting treatment of a RSV infection in a mammal or in a cell or alleviation or treatment of the symptoms of a RSV infection in the mammal. The instructional material of the kit of the invention may, for example, be affixed to a container which contains the composition of the invention or be shipped together with a container which contains the composition. Alternatively, the instructional material may be shipped separately from the container with the intention that the instructional material and the composition be used cooperatively by the recipient.

The invention also includes a kit for inhibition of RSV replication in a cell. The kit comprises a composition of the invention, which can be one or more compounds of the invention, or a pharmaceutical composition comprising one or more compounds of the invention or any combination thereof. The kit also includes an instructional material.

As used herein, inhibition of RSV replication in a cell means a reduction in RSV replication in a cell to a level lower than the level in an otherwise identical cell which was not administered the composition of the invention. Preferably, the reduction in RSV replication is by about 90 to about 99.9% relative to the otherwise identical cell which was not administered the composition of the invention. The level of RSV replication in a cell and therefore RSV load in a mammal that is also being assessed, can be assessed by any one of numerous methods known to the skilled artisan. For example, the level of RSV replication in a cell can be assessed by evaluating the number of RSV particles or amount of a viral component, such as a viral protein, a viral enzyme, or viral nucleic acid, in the cell or in fluid or debris associated with the cell. The number of infectious virus particles in a cell can be evaluated, for example, in a plaque assay. The level of a viral component such as a viral protein or enzyme in a cell can be evaluated using standard analytical techniques of protein biochemistry, such as, for example, using an activity assay for a viral enzyme, or using Western blotting or quantitative gel electrophoresis for a viral protein. Viral nucleic acid levels in a cell can be evaluated using standard analytical techniques such as Northern blotting and Southern Blotting or quantitation by polymerase chain reaction (PCR).

The invention includes methods for treatment of a RSV infection in a host. The methods comprise administering to the host one or more compounds of the invention, or any combination thereof, or a pharmaceutically acceptable salt thereof, in an amount effective to treat the virus infection. The compound may be administered by any of the methods described herein. Preferably, the host is a human.

The invention also includes methods of treating a RSV infection in a host by contacting the RSV in vitro, in vivo or ex-vivo with one or more compounds of the invention, or any combination thereof, or a pharmaceutically acceptable salt thereof, in an amount effective to treat the RSV infection (e.g. to inhibit virus replication, infectivity, life cycle processes or pathogenesis). Methods for testing the antiviral activity of a compound in-vitro are known to the skilled artisan, and are described, for example, in Kucera et al., 1990, AIDS Res. and Human Retrovir. 6:494.

The invention further includes methods of using one or more compounds of the invention, or any combination thereof, or a pharmaceutically acceptable salt thereof, in medical therapy (preferably for use in treating a virus infection) or for the manufacture of a medicament useful for the treatment of a virus infection.

The invention also includes methods of inhibiting RSV replication in a cell.

Any of the viral treatments described in the Background of the Invention can be used in combination or alternation with the compounds described in this specification. Nonlimiting examples include:
(1) an interferon and/or ribavirin (Battaglia, A. M. et al., Ann. Pharmacother. 34:487-494, 2000); Berenguer, M. et al. Antivir. Ther. 3(Suppl. 3):125-136, 1998);
(2) substrate-based NS3 protease inhibitors (Attwood et al., Antiviral peptide derivatives, PCT WO 98/22496, 1998; Attwood et al., Antiviral Chemistry and Chemotherapy 10.259-273, 1999; Attwood et al., Preparation and use of amino acid derivatives as anti-viral agents, German Patent Publication DE 19914474; Tung et al. Inhibitors of serine proteases, PCT WO 98/17679), including alphaketoamides and hydrazinoureas, and inhibitors that terminate in an electrophile such as a boronic acid or phosphonate. (Llinas-Brunet et al, PCT WO 99/07734).
(3) non-substrate-based inhibitors such as 2,4,6-trihydroxy-3-nitro-benzamide derivatives (Sudo K. et al., Biochemical and Biophysical Research Communications, 238:643-647, 1997; Sudo K. et al. Antiviral Chemistry and Chemotherapy 9:186, 1998), including RD3-4082 and RD3-4078, the former substituted on the amide with a 14 carbon chain and the latter processing a para-phenoxyphenyl group;
(4) thiazolidine derivatives which show relevant inhibition in a reverse-phase HPLC assay with an NS3/4A fusion protein and NS5A/5 substrate (Sudo K. et al., Antiviral Research 32:9-18, 1996), especially compound RD-1-6250, possessing a fused cinnamoyl moiety substituted with a long alkyl chain, RD4 6205 and RD4 6193;
(5) thiazolidines and benzanilides identified in Kakiuchi N. et al., J. FEBS Letters 421:217-220; Takeshita N. et al., Analytical Biochemistry 247:242-246, 1997;
(6) a phenanthrenequinone possessing activity against viral protease in a SDS-PAGE and autoradiography assay isolated from the fermentation culture broth of Streptomyces sp., Sch 68631 (Chu M. et al., Tetrahedron Letters 37:7229-7232, 1996), and Sch 351633, isolated from the fungus Penicillium griseofulvum, which demonstrates activity in a scintillation proximity assay (Chu M. et al., Bioorganic and Medicinal Chemistry Letters 9:1949-1952);
(7) selective NS3 inhibitors based on the macromolecule elgin c, isolated from leech (Qasim M. A. et al., iochemistry 36:1598-1607, 1997);
(8) antisense phosphorothioate oligodeoxynucleotides (S-ODN) complementary to sequence stretches in the 5′ non-coding region (NCR) of the virus (Alt M. et al., Hepatology 22:707-717, 1995), or nucleotides 326-348 comprising the 3′ end of the NCR and nucleotides 371-388 located in the core coding region of the IICV RNA (Alt M. et al., Archives of Virology 142:589-599, 1997; Galderisi U. et al., Journal of Cellular Physiology 181:251-257, 1999);
(9) inhibitors of IRES-dependent translation (Ikeda N et al., Japanese Patent Publication JP-08268890; Kai Y. et al. Prevention and treatment of viral diseases, Japanese Patent Publication JP-10101591);
(10) nuclease-resistant ribozymes. (Maccjak D. J. et al., Hepatology 30 Abstract 995, 1999); and
(11) other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.); and
(12) PEGASYS (pegylated interferon alfa-2a) by Roche, INFERGEN (interferon alfacon-1) by InterMune, OMNIFERON (natural interferon) by Viragen, ALUFERON by Human Genome Sciences, REIF (interferon beta-la) by Ares-Serono, Omega Interferon by BioMedicine, Oral Interferon Alpha by Amarillo Biosciences, Interferon gamma-b1 by InterMune, Interleukin-10 by Schering-Plough, IP-501 by Interneuron, Merimeodi VX-497 by Vertex, AMANTADINE (Symmetrel) by Endo Las Solvay, HEPTAZYME by RPI, IDN-6556 by Idun Pharma., XTL-002 by XTL., CIVACIR by NAI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

The invention will be further understood by the following non-limiting examples. These examples are provided for the purpose of illustration only and the invention is not limited to these examples, but rather includes all variations which are evident as a result of the teaching provided herein.

### 5.5 EXAMPLES

### Example 1

### Anti-RSV Activity of Selected Compounds

The ability of the active compounds to inhibit the growth of RSV was determined using a standard plaque assay with Hep-2 cell monolayers. The cell monolayers were infected with RSV (Long strain obtained from the American Tissue Culture Catalog, Cat# VR-26). After one hour of virus attachment, the cells were overlaid with medium containing methyl cellulose with or without serial concentrations of compound (0.4-50 μM). Each concentration was tested in triplicate.

After 6 days of incubation, the overlay medium was aspirated and the cells fixed with absolute ethanol and stained with crystal violet to visualize RSV plaques and cell cytotoxicity using a 4× dissecting microscope. To determine the percent inhibition of RSV plaque formation, the mean number of RSV plaques in the presence of compound was divided by the number of RSV plaques in the control without added compounds. The quotient was subtracted from 1 and then multiplied by 100% to establish the % inhibition. Results from these studies are shown in Table 1.

TABLE 1
Anti-RSV Activity

| | | | | 50% Endpoint (μM) | | |
|---|---|---|---|---|---|---|
| | | | | Effective Conc. | | |
| | | | | (EC₅₀)^{c} | Toxic Conc. | |
| | | | | RSV Plaque | (TC₅₀)^{c} | |
| Compound | R₁ | R₂ | R₃ | Count | Cell Growth | SI^{a} |
| KPC-15 | NHCOC₁₁H₂₃ | OCH₂CH₃ | PC^{b} | 2.4 μM | >100 μM | >42 |
| KPC-11 | NHCOC₉H₁₉ | OCH₂CH₃ | PC | 3.5 μM | >100 μM | >28 |

^{a}SI = Selectivity Index (TC50 Cell Growth divided by EC50 Plaque Inhibition).
^{b}PC = Phosphocholine [OPO₃⁻CH₂CH₂N⁺(CH₃)₃].
^{c}EC₅₀ and TC₅₀ determinations were calculated by the method of Chou et al cited by Piantadosi et al. (Piantadosi et al., 1991).
TC₅₀ was determined using radiolabeled thymidine incorporation.

As illustrated in Table 1,3-dodecylamido-2-ethoxypropylphosphocholine (KPC-15) and decylamido-2-ethoxypropylphosphocholine (KPC-11) had potent in vitro activity against RSV. The effectiveness of the compounds may be due to the chemical structure as a phosphocholine analog.

### Example 2

### Toxicity Tests

Cells in growth phase were treated with serial concentrations of compound for 48 hours and pulse labeled with 3H-thymidine for 6 hours. The cells were then harvested to measure total DNA synthesis in the presence or absence of the compound (Kucera et al., Antiviral Chemistry and Chemotherapy, 9: i57-i65 (1998)).

TC₅₀ determinations were calculated by the method of Chou et al. Elsvier: Amsterdam, 1985, as described in Piantadosi C. et al., Journal of Medicinal Chemistry, 34: 1408-1414 (1991). The TC₅₀ for KPC-15 and KPC-11 were unable to be determined since at the highest concentration evaluated (100 μM), 50% toxicity was not observed. Results from this study are also shown in Table 1 (above). Thus, KPC-15 and KPC-11, lacked significant toxicity in vitro with the TC₅₀>100 μM.

### Example 3

### Preparation of Pharmaceutical Compositions

Alkylamidophosphocholine compounds are synthesized as described in Ouyang et al., Journal of Medicinal Chemistry, 45 :2857-2866 (2002). The 3-alkylamido-2-alkoxypropylphosphocholine is obtained by reacting commercially available 3-amino-1,2,-propanediol with the appropriate acid chloride or anhydride. The primary alcohol is protected, and the secondary alcohol is alkylated with an alkyl bromide. The primary alcohol is deprotected and then reacted with 2-bromoethyl dichlorophosphate and trimethylamine to obtain the 3-alkylamido-2-alkoxypropylphosphocholine compound.

3-dodecylamido-2-ethoxypropylphosphocholine was synthesized as shown in FIG. 1, and as described in Ouyang et al. FIG. 1 describes the chemical synthesis of R, S, and racemic KPC-15 and shows there is a chiral center on the C-2 position of the three carbon backbone. 3-nonylamido-2-ethoxypropylphosphocholine also was synthesized according to this method.

A number of references have been cited, the entire disclosures of which are incorporated herein by reference.

1. A method for treating a host infected with RSV comprising administering an anti-RSV effective amount of a compound of Formula I: 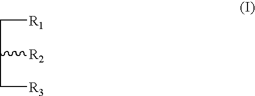
or a pharmaceutically acceptable salt or prodrug thereof,
wherein:
R₁ is selected from the group consisting of —NHC(O)Y, where Y is C₁-C₂₂ alkyl, C₂-C₂₂ alkenyl, and C₂-C₂₂ alkynyl;
R₂ is selected from the group consisting of —OX, where X is C₁-C₂₂ alkyl, C₂-C₂₂ alkenyl, C₂-C₂₂ alkynyl; and
R₃ is phosphocholine.

2. The method of claim 1 wherein Y and X are independently C₁-C₁₄ alkyl, C₂-C₁₄ alkenyl, or C₂-C₁₄ alkynyl.

3. The method of claim 1 wherein:
Y is —C₁₀H₂₁; and
X is —CH₂CH₃, —CH₂CH₂CH₃, —CH₂CH₂CH₂CH₃, or —C₁₀H₂₁.

4. The method of claim 1 wherein Y is —C₁₁H₂₃ and X is C₁-C₅ alkyl.

5. The method of claim 1 wherein Y is —C₉H₁₉ and X is C₉-C₁₁ alkyl.

6. The method of claim 1, wherein the compound is 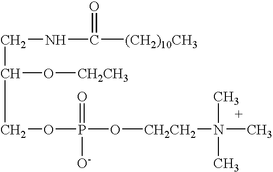
3-dodecanamido-2-ethoxypropyl-1-phosphocholine, 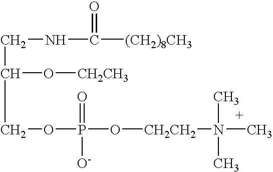
3-decanamido-2-ethoxypropyl-1-phosphocholine, 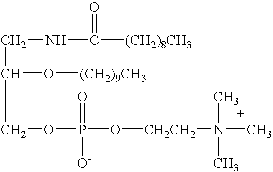
3-rdecanamido-2-decyloxypropyl-1-phosphocholine, 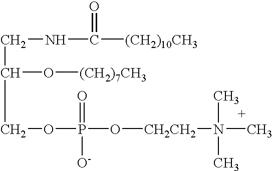
3-dodecanamido-2-octyloxypropyl-1-phosphocholine, 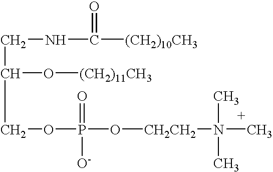
3-dodecanamido-2-dodecyloxypropyl-1-phosphocholine, or 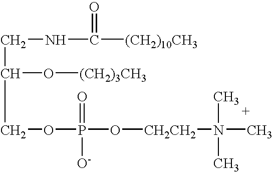
3-dodecanamido-2-butyloxy-1-phosphocholine; or a combination thereof.

7. The method of claim 1 wherein the host is a mammal.

8. The method of claim 1 wherein the host is a human.

9. A method for treating a host infected with RSV comprising administering an anti-RSV effective amount of a compound of Formula II: 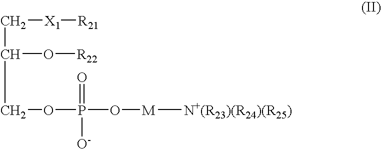
or a pharmaceutically acceptable salt or prodrugs thereof,
wherein:
M is C₂-C₄ alkyl;
X¹ is selected from the group consisting of —S—, —O—, —NH—, and —NHC(O)—;
R₂₁ is selected from the group consisting of C₁-C₂₀ straight chain alkyl, C₂-C₂₀ straight chain alkylene containing not more than four double bonds, and aryl;
R₂₂ is selected from the group consisting of C₁-C₂₀ straight chain alkyl, C₂-C₂₀ straight chain alkylene containing not more than four double bonds, and aryl; and
R₂₃, R₂₄, and R₂₅ are each independently selected from the group consisting of hydrogen, methyl, ethyl, propyl, and isopropyl.

10. The method of claim 9 wherein
M is —CH₂CH₂—;
X₁ is —NHC(O)—;
R₂₁ is selected from the group consisting of a C₁-C₁₆ straight chain alkyl and C₂-C₁₆ straight chain alkylene containing not more than one double bond;
R₂₂ is selected from the group consisting of a C₁-C₁₆ straight chain alkyl and C₂-C₁₆ straight chain alkylene containing not more than one double bond; and
R₂₃, R₂₄, and R₂₅ are each independently hydrogen or methyl.

11. The method of claim 9 wherein
R₂₁ is selected from the group consisting of C₁-C₁₆ straight chain alkyl and C₂-C₁₆ straight chain alkylene containing not more than one double bond; and
R₂₂ is selected from the group consisting of C₁-C₅ straight chain alkyl and C₂-C₅ straight chain alkylene containing not more than one double bond.

12. The method of claim 11 wherein R₂₁ is C₉-C₁₂ alkyl and R₂₂ is C₁-C₁₂ alkyl.

13. The method of claim 11 wherein R₂₁ is C₉-C₁₂ alkyl and R₂₂ is C₁-C₅ alkyl.

14. The method of claim 11 wherein R₂₁ is C₉-C₁₂ alkyl and R₂₂ is C₈-C₁₂ alkyl.

15. The method of claim 9 wherein the host comprises a mammal.

16. The method of claim 9 wherein the host comprises a human.

17. A method for treating a host infected with RSV comprising administering an anti-RSV effective amount of a compound of Formula III: 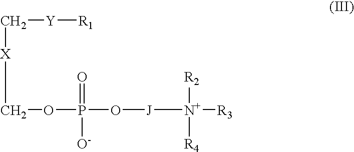
or a pharmaceutically acceptable salt or prodrug thereof,
wherein:
Y is selected from the group consisting of —S—, —O—, —NH—, —N(CH₃)—, —NHC(O)—, and —N(CH₃)C(O)—;
R₁ is selected from the group consisting of C₁-C₁₈ alkyl, C₂-C₁₈ alkenyl, C₂-C₁₈ alkynyl, and aryl;
X is a covalent bond or methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl), or —C(O)N(C₂-C₂₀ alkynyl);
J is a C₁-C₄ alkyl optionally substituted from one to three times with methyl or ethyl; and
R₂, R₃, and R₄ are independently hydrogen or C₁-C₃ alkyl.

18. The method of claim 17 wherein:
Y is —NHC(O)—;
R₁ is C₆-C₁₈ alkyl;
X is —C(H)(O—C₁-C₁₈ alkyl)- or —C(H)(O—C₁-C₁₈ alkenyl)-;
J is —CH₂CH₂—; and
R₂, R₃, and R₄ are each methyl.

19. The method of claim 18 wherein R₁ is —C₁₁H₂₃ and X is —C(H)(O—C₁-C₅ alkyl)- or —C(H)(O—C₁-C₅ alkenyl)-

20. The method of claim 18 wherein R₁ is —C₉H₁₉ and X is —C(H)(OC₂H₅)—.

21. The method of claim 17 wherein R₁ is —C₉H₁₉ and X is —C(H)(OC₁₀H₂₁)—.

22. The method of claim 17 wherein the host comprises a mammal.

23. The method of claim 17 wherein the host comprises a human.

24. A method for treating a host infected with RSV comprising administering an anti-RSV effective amount of a compound of Formula IV: 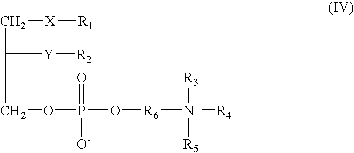
or a pharmaceutically acceptable salt or prodrug thereof,
wherein:
R₁ is selected from the group consisting of C₁-C₁₈ alkyl, C₂-C₁₈ alkenyl, and C₂-C₁₈ alkynyl that is optionally substituted from 1 to 5 times with —OH, —COOH, oxo, amino, or aryl;
X is selected from the group consisting of —NHC(O)—, —N(CH₃)C(O)—, —C(O)NH—, —C(O)N(CH₃)—, —S—, —S(O)—, —(SO₂)—, —O—, —NH—, and —N(CH₃)—;
R₂ is selected from the group consisting of C₁-C₁₄ alkyl, C₂-C₁₄ alkenyl, and C₂-C₁₄ alkynyl that is optionally substituted from 1 to 5 times with —OH, —COOH, oxo, amino, or aryl;
Y is selected from the group consisting of —NHC(O)—, —N(CH₃)C(O)—, —C(O)NH—, —C(O)N(CH₃)—, —S—, —S(O)—, —(SO₂)—, —O—, —NH—, —N(CH₃)—, and —OC(O)—;
R₆ is selected from the group consisting of C₂-C₆ alkyl; C₂-C₆ alkenyl, and C₂-C₆ alkynyl; and
R₃, R₄, and R₅ are independently methyl or ethyl, or R₃ and R₄ together form an aliphatic or heterocyclic ring having five or six ring atoms and R₅ is methyl or ethyl.

25. The method of claim 24 wherein:
R₂ is C₁-C₁₄ alkyl, C₂-C₁₄ alkenyl, or C₂-C₁₄ alkynyl;
R₆ is —CH₂CH₂—; and
R₃, R₄, and R₅ are each independently CH₃.

26. The method of claim 25 wherein R₂ is C₁-C₅ alkyl or C₂-C₅ alkenyl.

27. The method of claim 25 wherein R₁ is C₈-C₁₂ alkyl and R₂ is C₁-C₁₂ alkyl.

28. The method of claim 25 wherein R₁ is C₈-C₁₂ alkyl and R₂ is C₁-C₅ alkyl.

29. The method of claim 25 wherein R₁ is C₈-C₁₂ alkyl and R₂ is C₈-C₁₂ alkyl

30. The method of claim 27 wherein
X is —NHC(O), —N(CH₃)C(O)—, —C(O)NH—, —C(O)N(CH₃); and
Y is —O—, —NH—, or —N(CH₃)—.

31. The method of claim 24 wherein the host comprises a mammal.

32. The method of claim 24 wherein the host comprises a human.

33. The method of claim 24 wherein the compound comprises: 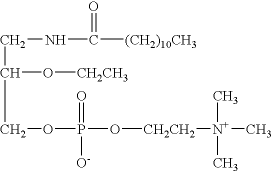
3-dodecanamido-2-ethoxypropyl-1-phosphocholine.

34. The method of claim 24 wherein the compound comprises: 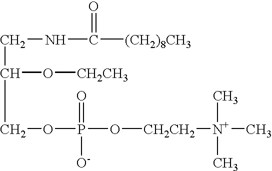
3-decanamido-2-ethoxypropyl-1-phosphocholine.

35. A method for treating a host infected with RSV comprising administering an anti-RSV effective amount of a compound of Formula AA-1: 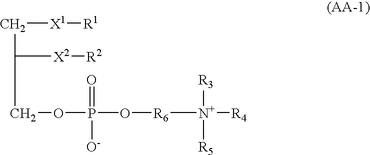
or a pharmaceutically acceptable salt or prodrug thereof,
wherein:
X¹ is —NHC(O)—;
X² is —O—;
R¹ is —C₁-C₂₂ alkyl;
R² is —C₁-C₂₂ alkyl;
R⁶ is —CH₂CH₂—; and
R³, R⁴, and R⁵ are methyl.

36. The method of claim 35, wherein
R¹ is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —CH₂)₁₀CH₃, —(CH₂)₁₁CH₃, —(CH₂)₁₂CH₃ or —(CH₂)₁₃CH₃; and
R²is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —CH₂)₁₀CH₃, —(CH₂)₁₁CH₃, —(CH₂)₁₂CH₃ or —(CH₂)₁₃CH₃.

37. The method of claim 36, wherein
R^{is —(CH}₂)₈CH₃, —(CH₂)₉CH₃, —(CH₂)₁₀CH₃, —(CH₂)₁₁CH₃; —(CH₂)₁₂CH₃, or —(CH₂)₁₃CH₃; and
R 2is CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, or —(CH₂)₇CH₃.

38. The method of claim 36, wherein
R¹ is —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, or —(CH₂)₁₂CH₃; and
R² is —(CH₂)₆CH₃, —(CH₂)₇CH₃, —(CH₂)₈CH₃, —(CH₂)₉CH₃CH₃, —(CH₂)₁₀CH₃, —(CH₂)₁₁CH₃, —(CH₂)₁₂CH₃, or —(CH₂)₁₃CH₃.

39. The method of claim 1, wherein the administering is orally, intravenously, parentally, intradermally, subcutaneously, topically, or by inhalation.
